(19) United States
(12) Patent Application Publication
(10) Pub. No.: US 2009/0247766 A1
(43) Pub. Date: Oct. 1, 2009

(54) Substituted Nitrogen Heterocycles and Synthesis and Uses Thereof

(75) Inventors: Nicos A. Petasis, Hacienda Heights, CA (US); Malgorzata Myslinska, Los Angeles, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/413,488

(22) Filed: Mar. 27, 2009

Related U.S. Application Data
(60) Provisional application No. 61/040,116, filed on Mar. 27, 2008.

(51) Int. Cl. C07D 409/04; C07D 495/04; C07D 407/04; C07D 209/10

(52) U.S. Cl. 548/425; 548/430; 548/453; 548/464; 548/510

(57) ABSTRACT

The invention relates to a nitrogen heterocycle compound of formula 1:

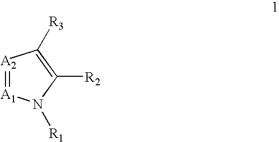

Also disclosed are a method of synthesizing the compound and use of the compound for treating various diseases and conditions.

### RELATED APPLICATION

The present application claims priority to U.S. Provisional Application Ser. No. 61/040,116, filed on Mar. 27, 2008, the content of which is incorporated herein by reference in its entirety.

### FIELD OF THE INVENTION

The invention pertains to nitrogen heterocycles and related derivatives, as well as methods for their synthesis and utilities of these compounds.

### BACKGROUND OF THE INVENTION

Nitrogen heterocycles containing nitrogen in the structure including pyrroles, indoles, aza-indoles, and their derivatives, are frequently present in many natural products and show a wide spectrum of biological properties. The indole ring, in particular, is arguably one of the most common heterocyclic structures present in nature and in synthetic therapeutics. Found in plant growth hormones such as indole-3-acetic acid, alkaloids (gramines, ergines), tryptamines (melatonin and serotonin), amino acids (tryptophan) and many therapeutic agents (e.g., Indomethacin, Sumatripan, and Fluvastatin), the indole skeleton exhibits a wide range of biological and pharmaceutical activities.

Indoles and related N-heterocyclic derivatives have been used as synthetic intermediates, and as therapeutic agents for a plethora of diseases and conditions. They are anti-inflammatory agents, antimalarial agents, antifungal agents, antibacterial agents, antiviral agents, antimycotic agents, anticancer agents, antitumor agents, antidepressants, agents for treating seasonal affective disorder, agents for treating premenstrual dysphoric disorder, selective serotonin reuptake inhibitors or agonists, tryptophan mimics, DNA topoisomerase I inhibitors, cancer chemotherapy agents, kinase inhibitors, immunomodulatory agents, antihypertensive agents, plant growth regulating hormones, neurotransmitters, antiprotozoal agents, antimigraine agents, sPLA2 inhibitors, MCP-1 inhibitors, glycogen phosphorylase inhibitors, platelet activating factor (PAF) inhibitors, allosteric enhancers of adenosine receptors, tyrosine kinase inhibitors, GnRH antagonists, tranquillizers, antiangiogenic agents, agents for treating rheumatoid arthritis, osteoarthritis, sepsis, asthma, adult respiratory distress syndrome, cardiovascular disorders, and Alzheimer's disease, allosteric inhibitors of the hepatitis C virus, antiplasmodial agents, cytotoxic agents, DNA intercalators, MDM2 inhibitors, HIV integrase inhibitors, molecules that target the ligand-binding pocket of PDZ domains of NHERF1 multi-functional adaptor protein, neuroprotective agents, agents for the treatment of liver disease, cirrhosis, hepatocellular carcinoma, chronic hepatitis, and other diseases and conditions.

As a consequence of the importance of the indole moiety, a large and growing number of methods have been developed for their synthesis over the years. The most important methodologies include the Fischer indole synthesis, the Leimgruber-Batcho synthesis, the Bischler-Möhlau indole synthesis, the Gassman method, the Nenitzescu indole synthesis, the Madelung cyclization, and various palladium catalyzed cyclizations.

Despite the availability of a variety of methods for the synthesis of indoles and related N-heterocycles, such methods are often not suitable for the efficient synthesis of numerous types of substituted nitrogen heterocycle derivatives, while many such compounds are not know in the art, due to the lack of suitable methods for their synthesis. Hence, there is a need for the development of conceptually new and efficient methods for the synthesis of such molecules from simple and readily available precursors. Such methods can lead not only to improved methods for the synthesis of known types of such heterocycles, but also to the availability of previously unknown members of this class of compounds.

### SUMMARY OF THE INVENTION

This invention relates to novel substituted N-heterocyclic compounds, novel methods for synthesizing substituted N-heterocyclic compounds, and utilities of substituted N-heterocyclic compounds.

In one aspect, the invention features a nitrogen heterocycle compound of formula 1:

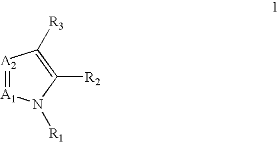

wherein:
R₁ is selected from the group consisting of H, alkyl, allyl, aryl, heteroaryl, acyl, trifluoroacyl, arylacyl, heteroarylacyl, pent-4-enylacyl, alkoxyacyl, allyloxyacyl, aryloxyacyl, aminoacyl, alkylaminoacyl, dialkylaminoacyl, arylmethyl, triarylmethyl, alkylsulfinyl, arylsulfinyl, alkylsulfonyl, arylsulfonyl, trialkylsilyl, aryldialkylsilyl, diarylalkylsilyl, bis(trimethylsilyl)methyl, and trialkylsilyl-ethanesulfonyl;
R₂ is selected from the group consisting of H, alkyl, allyl, alkenyl, alkynyl, allenyl, aryl, heteroaryl, trifluoromethyl, difluoromethyl, fluooroalkyl, difluoroalkyl, trifluoroalkyl, polyfluoroalkyl, acyl, carboxyl, alkoxyacyl, aryloxyacyl, aminoacyl, alkylaminoacyl, dialkylaminoacyl, and arylmethyl;
R₃ is selected from the group consisting of H, alkyl, allyl, aryl, heteroaryl, trifluoromethyl, 2,2,2-trifluoroethyl, fluooroalkyl, difluoroalkyl, trifluoroalkyl, polyfluoroalkyl, acyl, trifluoroacyl, arylacyl, carboxyl, alkoxyacyl, aryloxyacyl, aminoacyl, alkylaminoacyl, dialkylaminoacyl, amino, acylamino, alkoxyacylamino, aminoacylamino, alkylamino, dialkylamino, arylamino, arylalkylamino, and diarylamino;
A₁ and A₂ are independently selected from the group consisting of N and C—R, wherein R is selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, aminoacyl, alkylaminoacyl, dialkylaminoacyl, fluoro, bromo, iodo, hydroxy, alkoxy, aryloxy, cyano, amino, alkylamino, dialkylamino, arylamino, arylalkylamino, and diarylamino, and wherein groups A₁ and A₂ can be joined together to form a carbocyclic, heterocyclic, aromatic, or heteroaromatic ring; and
any two of R₁-R₃ and A₁-A₂ can be joined together to form a carbocyclic, heterocyclic, aromatic, or heteroaromatic ring.

In some embodiments, a compound of the invention is selected from the group consisting of compounds 2-29:

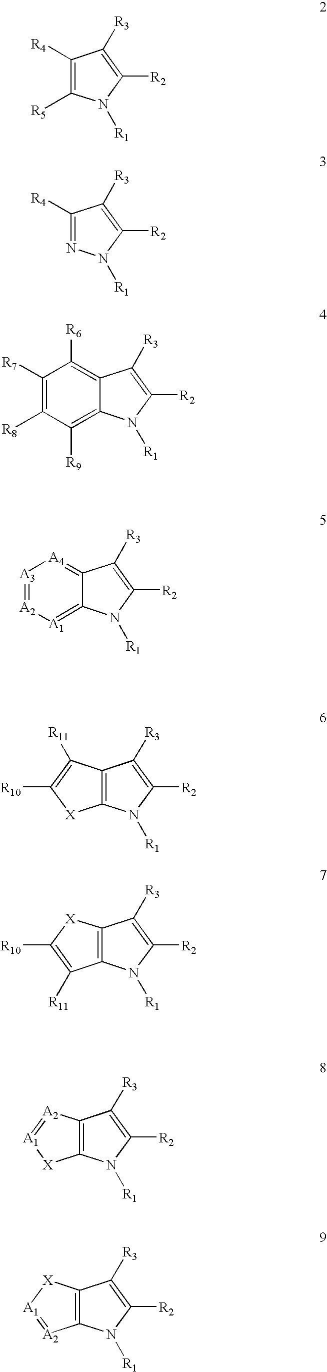

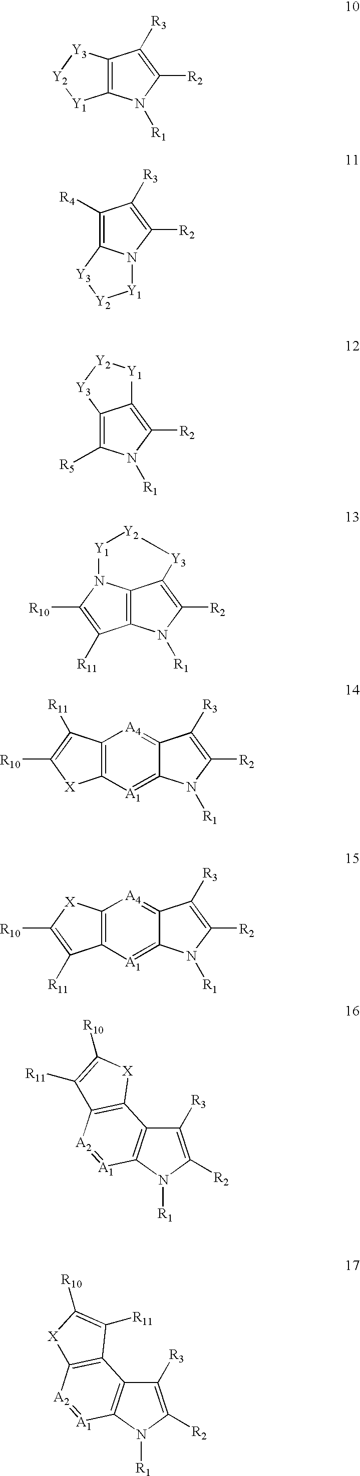

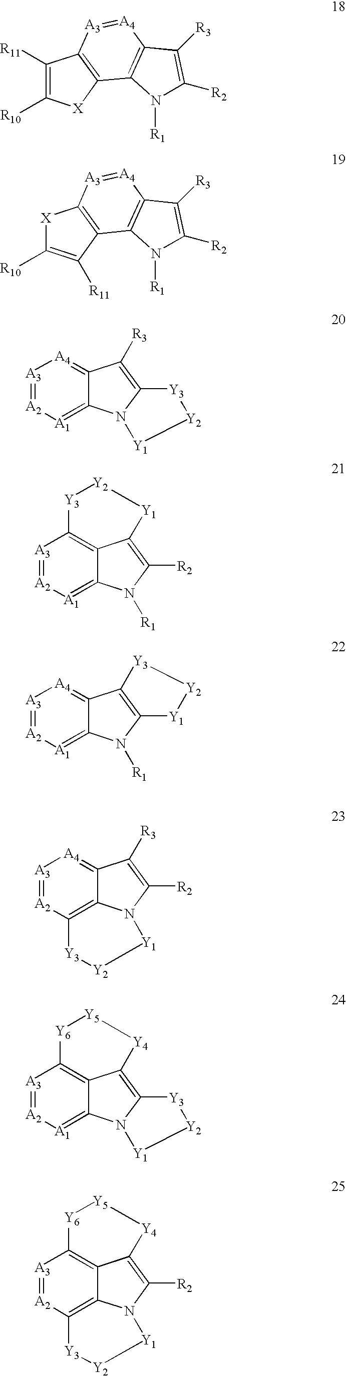

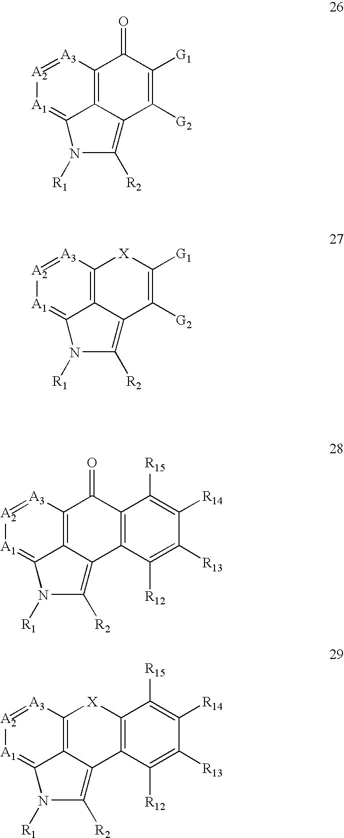

wherein:
R₁-R₃ and A₁-A₂ are defined as above;
R₄, R₆-R₉, and R₁₁-R₁₅ are independently selected from the group consisting of H, alkyl, allyl, aryl, heteroaryl, trifluoromethyl, 2,2,2-trifluoroethyl, fluooroalkyl, difluoroalkyl, trifluoroalkyl, polyfluoroalkyl, acyl, trifluoroacyl, arylacyl, carboxyl, alkoxyacyl, aryloxyacyl, fluoro, chloro, bromo, iodo, hydroxy, alkoxy, aryloxy, cyano, amino, acylamino, alkoxyacylamino, aminoacylamino, alkylamino, dialkylamino, arylamino, arylalkylamino, and diarylamino;
R₅ and R₁₀ are independently selected from the group consisting of H, alkyl, allyl, alkenyl, alkynyl, allenyl, aryl, heteroaryl, trifluoromethyl, fluooroalkyl, difluoroalkyl, trifluoroalkyl, polyfluoroalkyl, acyl, trifluoroacyl, arylacyl, carboxyl, alkoxyacyl, aryloxyacyl, fluoro, chloro, bromo, iodo, acyl, carboxyl, alkoxyacyl, aryloxyacyl, aminoacyl, alkylaminoacyl, arylaminoacyl, and dialkylaminoacyl;
A₃-A₄ are independently selected from the group consisting of N and C—R, wherein R is selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, fluoro, chloro, bromo, iodo, hydroxy, alkoxy, aryloxy, cyano, amino, alkylamino, dialkylamino, arylamino, arylalkylamino, diarylamino, aminoacyl, alkylaminoacyl, arylaminoacyl, and dialkylaminoacyl, and wherein there are no more than two Ns among A₁, A₂, A₃, and A₄, and wherein any two of A₁-A₄ can be joined together to form a carbocyclic, heterocyclic, aromatic, or heteroaromatic ring;
X is selected from the group consisting of O, S, and NRa, wherein Ra is selected from the group consisting of H, alkyl, allyl, aryl, heteroaryl, acyl, trifluoroacyl, arylacyl, heteroarylacyl, pent-4-enylacyl, alkoxyacyl, allyloxyacyl, aryloxyacyl, aminoacyl, alkylaminoacyl, dialkylaminoacyl, arylmethyl, triarylmethyl, alkylsulfinyl, arylsulfinyl, alkylsulfonyl, arylsulfonyl, alkylsulfinyl, arylsulfinyl, alkylsulfonyl, arylsulfonyl, trialkylsilyl, aryldialkylsilyl, diarylalkylsilyl, bis(trimethylsilyl)-methyl, and trialkylsilylethanesulfonyl;
Y₁-Y₂-Y₃ and Y₄-Y₅-Y₆ are independently a chain of 3-20 atoms selected from the group consisting of carbon, nitrogen, oxygen, and sulfur atoms;
G₁ and G₂ are independently selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, aminoacyl, alkylaminoacyl, dialkylaminoacyl, fluoro, bromo, iodo, hydroxy, alkoxy, aryloxy, cyano, amino, alkylamino, dialkylamino, arylamino, arylalkylamino, and diarylamino, and wherein G₁ and G₂ can be joined together to form a carbocyclic, heterocyclic, aromatic, or heteroaromatic ring; and
any two of R₁-R₁₅, A₁-A₄, and G₁-G₂ can be joined together to form a carbocyclic, heterocyclic, aromatic, or heteroaromatic ring.

In some embodiments, the Y₁-Y₂-Y₃ or Y₄-Y₅-Y₆ chain contains one or more substituents, including embedded keto, alkenyl, alkynyl, aryl, or heteroaryl groups.

In some embodiments, R₂ and R₃ are independently selected from the fluorine-containing groups consisting of fluooroalkyl, difluoroalkyl, trifluoroalkyl, polyfluoroalkyl, fluoroaryl, fluoroheteroaryl, fluorocycloalkyl, and fluoroheterocyclic.

In another aspect, the invention features a method for the synthesis of a compound of the invention. The method comprises:
(a) providing an amino acid of formula 30:

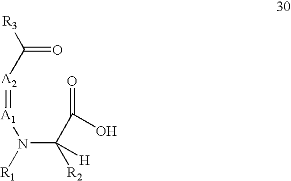

wherein R₁-R₃ and A₁-A₂ are defined as above; and
(b) reacting the amino acid of formula 30 with an acid activator and a base to form a compound of the invention.

In some embodiments, the amino acid of compound 30 is converted to an intermediate of formula 31-34, which is transformed to compound 1:

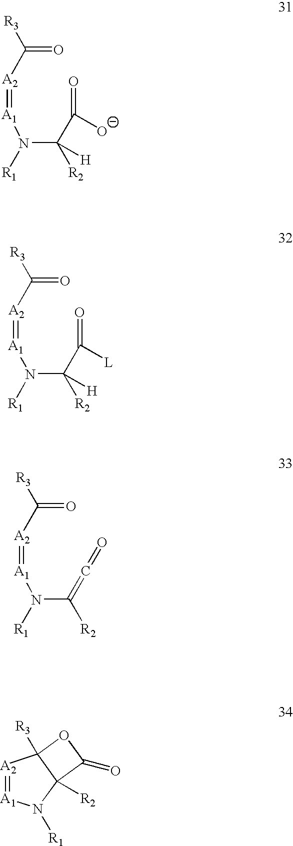

wherein:
R₁-R₃ and A₁-A₂ are defined as above; and
L is chloro, bromo, iodo, fluoro, OR′, OC(═O)R′, OC(═O)OR′, OC(═O)NR″R′″, OS(═O)R′, OSO₂R′, OPO₂R′, OPO₂OR′, or OP(═O)OR′,
wherein R′ is alkyl, fluoroalkyl, aryl, heteroaryl, or 2-N-alkyl-pyridinium, and R″ and R′″ are independently H, alkyl, or aryl.

In some embodiments, the acid activator is selected from the group consisting of an anhydride R^{L}C(═O)—O—C(═O)R^{L}, an acyl fluoride R^{L}C(═O)F, an acyl chloride R^{L}C(═O)Cl, an acyl bromide R^{L}C(═O)Br, a sulfinyl chloride R^{L}S(═O)Cl, a sulfonyl chloride R^{L}SO₂Cl, a sulfinyl anhydride R^{L}S(═O)—O—S(═O)R^{L}, a sulfonyl anhydride R^{L}SO₂—O—SO₂R^{L}, a chloroformate R^{L}OC(═O)Cl, an alkoxyacyl anhydride R^{L}OC(═O)—O—C(═O)OR^{L}, a phosphoryl chloride R^{L}P(═O)Cl, a phosphinyl chloride R^{L}R^{L}P(═O)Cl, a 2-halo-N-alkyl-pyridinium salt, N,N-dimethylphosphoramidic dichloride, thionyl chloride, and oxalyl chloride, wherein R^{L} is methyl, trifluoromethyl, alkyl, fluoroalkyl, difluoroalkyl, trifluoroalkyl, aryl, nitroaryl, or heteroaryl.

In some embodiments, the base is selected from the group consisting of dialkylamine, trialkylamine, and an N-heterocyclic compound containing a basic N-atom. The N-heterocyclic compound containing a basic N-atom may be selected from the group consisting of pyridine, lutidine, quinoline, isoquinoline, imidazole, diazabicycloundecane (DBU), diazabicyclononane (DBN), and 1,4-diazabicyclo[2.2.2]octane (DABCO).

In some embodiments, the compound synthesized is a compound of formula 4:

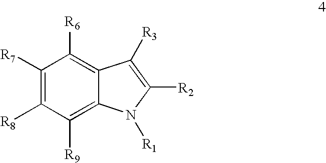

wherein R₁-R₃ and R₆-R₉ are defined as above.

The amino acid of formula 30 may be prepared in one step by the reaction of an amine compound of formula 35, a boron compound of formula 36 or 37, and glyoxylic acid of formula 38 or its hydrated form:

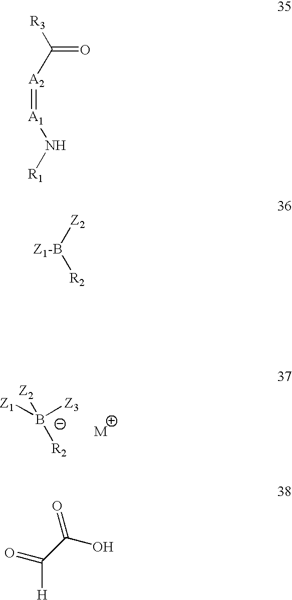

wherein
R₁-R₃ and A₁-A₂ are defined as above;
Z₁-Z₃ are independently selected from the group consisting of hydroxy, alkoxy, acyloxy, fluoro, chloro, bromo, alkylamino, and arylamino; and
M is potassium or tetralkylamino.

In some embodiments, the amine compound of formula 35 is an aniline.

In some embodiments, the boron compound of formula 36 is an organoboronic acid or boronate.

In some embodiments, the boron compound of formula 37 is an organotrifluoroborate salt.

In some embodiments, at least one of R₂ and R₃ is selected from the fluorine-containing groups consisting of fluooroalkyl, difluoroalkyl, trifluoroalkyl, polyfluoroalkyl, fluoroaryl, fluoroheteroaryl, fluorocycloalkyl, and fluoroheterocyclic.

A compound synthesized according to a method of the invention is within the invention.

The compounds of the invention can be used as anti-inflammatory agents, antimalarial agents, antifungal agents, antibacterial agents, antiviral agents, antimycotic agents, anticancer agents, antitumor agents, antidepressants, agents for treating seasonal affective disorder, agents for treating premenstrual dysphoric disorder, selective serotonin reuptake inhibitors or agonists, tryptophan mimics, DNA topoisomerase I inhibitors, cancer chemotherapy agents, kinase inhibitors, immunomodulatory agents, antihypertensive agents, plant growth regulating hormones, neurotransmitters, antiprotozoal agents, antimigraine agents, sPLA2 inhibitors, MCP-1 inhibitors, glycogen phosphorylase inhibitors, platelet activating factor (PAF) inhibitors, allosteric enhancers of adenosine receptors, tyrosine kinase inhibitors, GnRH antagonists, tranquillizers, antiangiogenic agents, agents for treating rheumatoid arthritis, osteoarthritis, sepsis, asthma, adult respiratory distress syndrome, cardiovascular disorders, Alzheimer's disease, allosteric inhibitors of the hepatitis C virus, antiplasmodial agents, cytotoxic agents, DNA intercalators, MDM2 inhibitors, HIV integrase inhibitors, molecules that target the ligand-binding pocket of PDZ domains of NHERF1 multi-functional adaptor proteins, neuroprotective agents, agents for the treatment of liver disease, cirrhosis, hepatocelular carcinoma, chronic hepatitis, and other related diseases and conditions.

Accordingly, the invention provides a method of administering to a subject in need thereof an effective amount of a compound of the invention.

Furthermore, the compounds of the invention can also be used as fluorescent dyes.

The above-mentioned and other features of this invention and the manner of obtaining and using them will become more apparent, and will be best understood, by reference to the following description.

### DETAILED DESCRIPTION OF THE INVENTION

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, the nomenclature alkyl, alkoxy, carbonyl, etc. is used as is generally understood by those of skill in this art. As used in this specification, alkyl groups can include straight-chained, branched and cyclic alkyl radicals containing up to about 20 carbons, or 1 to 16 carbons, and are straight or branched. Exemplary alkyl groups herein include, but are not limited to, methyl, ethyl, propyl, isopropyl, isobutyl, n-butyl, sec-butyl, tert-butyl, isopentyl, neopentyl, tert-pentyl, and isohexyl. As used herein, lower alkyl refer to carbon chains having from about 1 or about 2 carbons up to about 6 carbons. Suitable alkyl groups may be saturated or unsaturated. Further, an alkyl may also be substituted one or more times on one or more carbons with substituents selected from a group consisting of C1-C15 alkyl, allyl, allenyl, alkenyl, C3-C7 heterocycle, aryl, halo, hydroxy, amino, cyano, oxo, thio, alkoxy, formyl, carboxy, carboxamido, phosphoryl, phosphonate, phosphonamido, sulfonyl, alkylsulfonate, arylsulfonate, and sulfonamide. Additionally, an alkyl group may contain up to 10 heteroatoms, in certain embodiments, 1, 2, 3, 4, 5, 6, 7, 8 or 9 heteroatom substituents. Suitable heteroatoms include nitrogen, oxygen, sulfur, and phosphorous.

As used herein, “cycloalkyl” refers to a mono- or multicyclic ring system, in certain embodiments of 3 to 10 carbon atoms, in other embodiments of 3 to 6 carbon atoms. The ring systems of the cycloalkyl group may be composed of one ring or two or more rings which may be joined together in a fused, bridged or spiro-connected fashion. As used herein, “aryl” refers to aromatic monocyclic or multicyclic groups containing from 3 to 16 carbon atoms. As used in this specification, aryl groups are aryl radicals which may contain up to 10 heteroatoms, in certain embodiments, 1, 2, 3, or 4 heteroatoms. An aryl group may also be optionally substituted one or more times, in certain embodiments, 1 to 3 or 4 times with an aryl group or a lower alkyl group and it may be also fused to other aryl or cycloalkyl rings. Suitable aryl groups include, for example, phenyl, naphthyl, tolyl, imidazolyl, pyridyl, pyrroyl, thienyl, pyrimidyl, thiazolyl, and furyl groups. As used in this specification, a ring is defined as having up to 20 atoms that may include one or more nitrogen, oxygen, sulfur, or phosphorous atoms, provided that the ring can have one or more substituents selected from the group consisting of hydrogen, alkyl, allyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, phosphonate, phosphonamido, and sulfonyl, and further provided that the ring may also contain one or more fused rings, including carbocyclic, heterocyclic, aryl or heteroaryl rings. As used herein, alkenyl and alkynyl carbon chains, if not specified, contain from 2 to 20 carbons, or 2 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl, and isoquinolinyl. As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen, or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen may be quaternized to form an ammonium group where the substituents are selected as above. As used herein, “alkoxy” refers to RO—, in which R is alkyl, including lower alkyl. As used herein, “aryloxy” refers to RO—, in which R is aryl, including lower aryl, such as phenyl.

The invention provides novel substituted N-heterocyclic compounds and methods for their synthesis.

The first aspect of the invention relates to new N-heterocycles with novel substitution patterns, including fused ring systems such as aromatic rings, hetero-aromatic rings, carbocyclic rings, and heterocyclic rings containing oxygen, nitrogen, and sulfur atoms.

The invention provides N-heterocycles of formula 1:

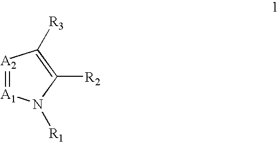

wherein:
R₁ is selected from the group consisting of H, alkyl, allyl, aryl, heteroaryl, acyl, trifluoroacyl, arylacyl, heteroarylacyl, pent-4-enylacyl, alkoxyacyl, allyloxyacyl, aryloxyacyl, aminoacyl, alkylaminoacyl, dialkylaminoacyl, arylmethyl, triarylmethyl, alkylsulfinyl, arylsulfinyl, alkylsulfonyl, arylsulfonyl, trialkylsilyl, aryldialkylsilyl, diarylalkylsilyl, bis(trimethylsilyl)methyl, and trialkylsilyl-ethanesulfonyl;
R₂ is selected from the group consisting of H, alkyl, allyl, alkenyl, alkynyl, allenyl, aryl, heteroaryl, trifluoromethyl, difluoromethyl, fluooroalkyl, difluoroalkyl, trifluoroalkyl, polyfluoroalkyl, acyl, carboxyl, alkoxyacyl, aryloxyacyl, aminoacyl, alkylaminoacyl, dialkylaminoacyl, and arylmethyl;
R₃ is selected from the group consisting of H, alkyl, allyl, aryl, heteroaryl, trifluoromethyl, 2,2,2-trifluoroethyl, fluooroalkyl, difluoroalkyl, trifluoroalkyl, polyfluoroalkyl, acyl, trifluoroacyl, arylacyl, carboxyl, alkoxyacyl, aryloxyacyl, aminoacyl, alkylaminoacyl, dialkylaminoacyl, amino, acylamino, alkoxyacylamino, aminoacylamino, alkylamino, dialkylamino, arylamino, arylalkylamino, and diarylamino;
A₁ and A₂ are independently selected from the group consisting of N and C—R, wherein R is selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, aminoacyl, alkylaminoacyl, dialkylaminoacyl, fluoro, bromo, iodo, hydroxy, alkoxy, aryloxy, cyano, amino, alkylamino, dialkylamino, arylamino, arylalkylamino, and diarylamino, and wherein groups A₁ and A₂ can be joined together to form a carbocyclic, heterocyclic, aromatic, or heteroaromatic ring; and
any two of R₁-R₃ and A₁-A₂ can be joined together to form a carbocyclic, heterocyclic, aromatic, or heteroaromatic ring.

In particular, the invention provides N-heterocycles selected from the group consisting of compounds of the general formula 2-29:

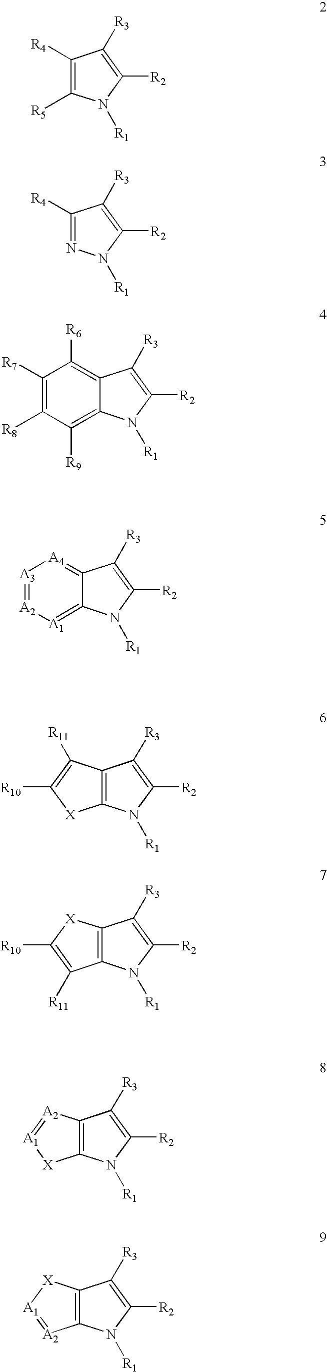

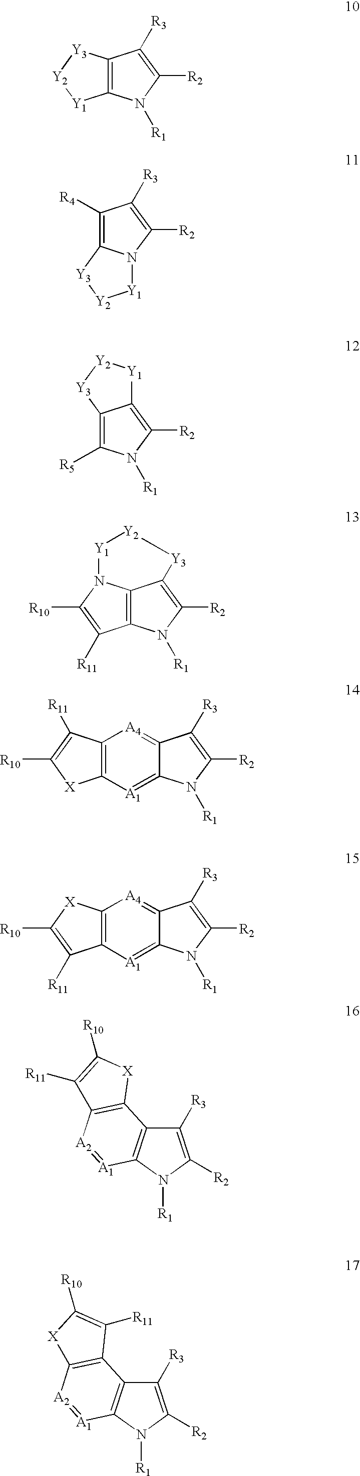

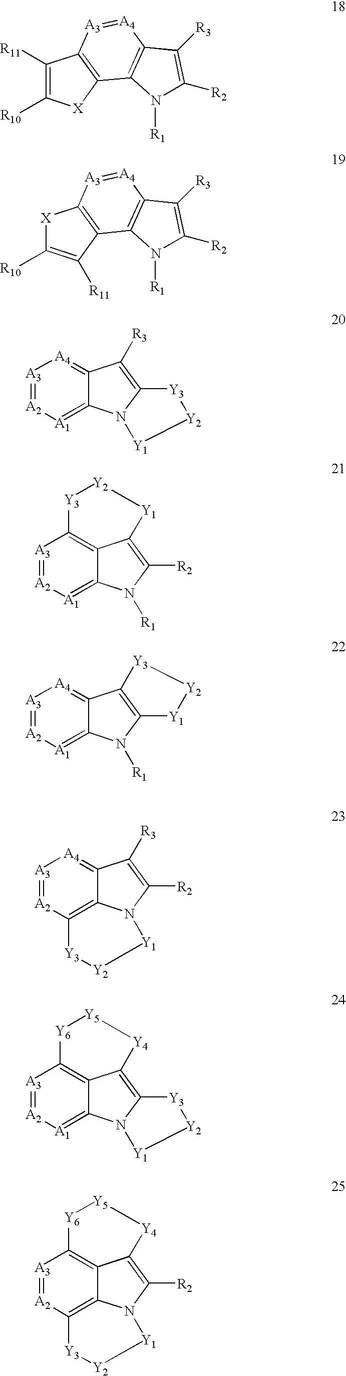

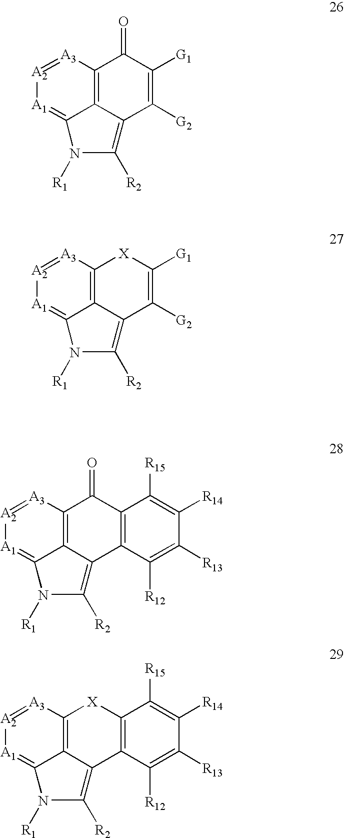

wherein:
R₁-R₃ and A₁-A₂ are defined as above;
R₄, R₆-R₉, and R₁₁-R₁₅ are independently selected from the group consisting of H, alkyl, allyl, aryl, heteroaryl, trifluoromethyl, 2,2,2-trifluoroethyl, fluooroalkyl, difluoroalkyl, trifluoroalkyl, polyfluoroalkyl, acyl, trifluoroacyl, arylacyl, carboxyl, alkoxyacyl, aryloxyacyl, fluoro, chloro, bromo, iodo, hydroxy, alkoxy, aryloxy, cyano, amino, acylamino, alkoxyacylamino, aminoacylamino, alkylamino, dialkylamino, arylamino, arylalkylamino, and diarylamino;
R₅ and R₁₀, are independently selected from the group consisting of H, alkyl, allyl, alkenyl, alkynyl, allenyl, aryl, heteroaryl, trifluoromethyl, fluooroalkyl, difluoroalkyl, trifluoroalkyl, polyfluoroalkyl, acyl, trifluoroacyl, arylacyl, carboxyl, alkoxyacyl, aryloxyacyl, fluoro, chloro, bromo, iodo, acyl, carboxyl, alkoxyacyl, aryloxyacyl, aminoacyl, alkylaminoacyl, arylaminoacyl, and dialkylaminoacyl;
A₃-A₄ are independently selected from the group consisting of N and C—R, wherein R is selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, fluoro, chloro, bromo, iodo, hydroxy, alkoxy, aryloxy, cyano, amino, alkylamino, dialkylamino, arylamino, arylalkylamino, diarylamino, aminoacyl, alkylaminoacyl, arylaminoacyl, and dialkylaminoacyl, and wherein there are no more than two Ns among A₁, A₂, A₃, and A₄, and wherein any two of A₁-A₄ can be joined together to form a carbocyclic, heterocyclic, aromatic, or heteroaromatic ring;
X is selected from the group consisting of O, S, and NRa, wherein Ra is selected from the group consisting of H, alkyl, allyl, aryl, heteroaryl, acyl, trifluoroacyl, arylacyl, heteroarylacyl, pent-4-enylacyl, alkoxyacyl, allyloxyacyl, aryloxyacyl, aminoacyl, alkylaminoacyl, dialkylaminoacyl, arylmethyl, triarylmethyl, alkylsulfinyl, arylsulfinyl, alkylsulfonyl, arylsulfonyl, alkylsulfinyl, arylsulfinyl, alkylsulfonyl, arylsulfonyl, trialkylsilyl, aryldialkylsilyl, diarylalkylsilyl, bis(trimethylsilyl)-methyl, and trialkylsilylethanesulfonyl;
Y₁-Y₂-Y₃ and Y₄-Y₅-Y₆ are independently a chain of 3-20 atoms selected from the group consisting of carbon, nitrogen, oxygen, and sulfur atoms, provided that this chain can contain one or more substituents, including embedded keto, alkenyl, alkynyl, aryl, or heteroaryl groups;
G₁ and G₂ are independently selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, aminoacyl, alkylaminoacyl, dialkylaminoacyl, fluoro, bromo, iodo, hydroxy, alkoxy, aryloxy, cyano, amino, alkylamino, dialkylamino, arylamino, arylalkylamino, and diarylamino, and wherein G₁ and G₂ can be joined together to form a carbocyclic, heterocyclic, aromatic, or heteroaromatic ring; and
any two of R₁-R₁₅, A₁-A₄, and G₁-G₂ can be joined together to form a carbocyclic, heterocyclic, aromatic, or heteroaromatic ring.

In the second aspect, the invention provides a method for the synthesis of the provided compounds. In one embodiment, the method involves the preparation of said compounds from an appropriate amino acid precursor. The amino acid precursor is activated by any of those amino acid activators known in the art and as a result an intermediate ketene is formed which subsequently reacts with the ketone moiety in an intramolecular fashion. This method is suitable for the synthesis of the new compounds provided by this invention. It is also suitable for the improved synthesis of known types of N-heterocycles.

In one embodiment, the amino acid precursors can be provided by the one step reaction among an aniline, a keto acid, and an organoboron derivative, including organoboronic acid, boronate, or trifluoroborates. In other embodiments, the amino acid derivative can be prepared by methods known in the art.

More specifically, the invention provides a method for the synthesis of compounds of formula 1. The provided method involves the preparation of compounds of formula 1 directly from amino acid precursor of formula 30. Compound 30 is treated with a base and an acid activator to give compound 1 with a loss of carbon dioxide. Presumably during the provided reaction conditions compound 30 is converted to compound 1 via intermediates of the general formula 31-34.

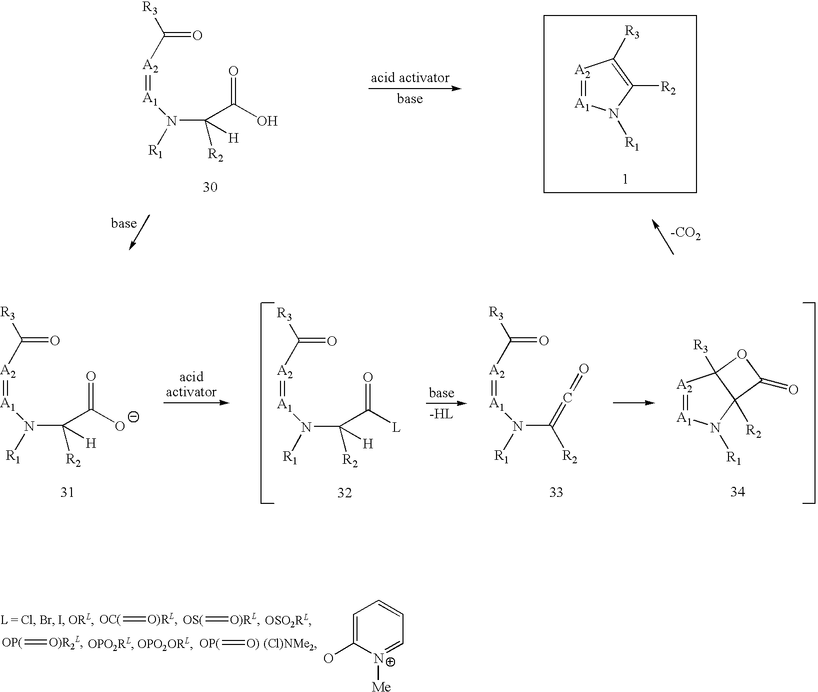

Under these conditions, compound 30 is converted to the corresponding salt 31, which can also be used directly in the reaction. Upon treatment with the acid activator in the presence of base, compound 30 or compound 31 is converted to intermediate compound 32 having a leaving group L, selected from the group consisting of chloro, bromo, iodo, alkoxy, aryloxy, acyloxy, acetoxy, arylacyloxy, alkylacyloxy, trifluoromethyl-acyloxy, difluoromethylacyloxy, alkylsulfinyloxy, arylsufinyloxy, alkylsulfonyloxy, arylsulfonyloxy, arylphosphinyloxy, arylphosphoryloxy, diarylphosphoryloxy, dialkylaminochlorophosphoramidyloxy, and N-alkylpyridinium-2-alkoxy.

In some embodiments, the provided acid activator is selected from the group consisting of an anhydride R^{L}C(═O)—O—C(═O)R^{L}, an acyl fluoride R^{L}C(═O)F, an acyl chloride R^{L}C(═O)Cl, an acyl bromide R^{L}C(═O)Br, a sulfinyl chloride R^{L}S(═O)Cl, a sulfonyl chloride R^{L}SO₂Cl, a sulfinyl anhydride R^{L}S(═O)—O—S(═O)R^{L}, a sulfonyl anhydride R^{L}SO₂—O—SO₂R^{L}, a chloroformate R^{L}OC(═O)Cl, an alkoxyacyl anhydride R^{L}OC(═O)—O—C(═O)OR^{L}, a phosphoryl chloride R^{L}P(═O)Cl, a phosphinyl chloride R^{L}R^{L}P(═O)Cl, a 2-halo-N-alkyl-pyridinium salt, N,N-dimethylphosphoramidic dichloride, thionyl chloride, and oxalyl chloride, wherein R^{L} is methyl, trifluoromethyl, alkyl, fluoroalkyl, difluoroalkyl, trifluoroalkyl, aryl, nitroaryl, or heteroaryl.

The preferred acid activators for the provided method include the following: acetic anhydride (Ac₂O), trifluoroacetic anhydride (CF₃CO)₂O, other carboxylic acid anhydrides (RCO)₂O, acetyl chloride, benzoyl chloride, other acyl halides (RCOX, where X=Cl, Br, or F), sulfonyl halides such as mesyl chloride, tosyl chloride, nosyl chloride, trifluoromethylsulfonyl chloride, trifluoromethylsulfonyl anhydride, alkyl chloroformates, Boc anhydride, thionyl chloride, and oxalyl chloride.

Upon reaction with a base, compound 32 is converted in situ to a ketene intermediate 33, which reacts intramolecularly with a carbonyl to form the β-lactone intermediate 34 that undergoes in situ fragmentation with the loss of carbon dioxide to form the product 1. Overall, compound 30 is converted directly to product 1, without any isolation or characterization of intermediates 31-34.

The type of base that can be used in the provided method include the following: trialkyl amines such as triethyl amine, diisoproyl ethyl amine, and N-heterocyclic compounds containing a basic N-atom, such as pyridines, lutidines, quinolines, isoquinolines, imidazoles, diazabicycloundecane (DBU), diazabicyclononane (DBN), and 1,4-diazabicyclo[2.2.2]octane (DABCO).

In specific embodiments, different types of amino acid precursors are treated with an amino acid activator and a base to form the provided N-heterocycles, compounds 2-29. Depending on the substituents and ring systems present on the amino acid precursor, a variety of novel N-heterocycles can be made with the present invention.

The required amino acid compounds of formula 30 can be prepared via a variety of methods known in the art.

In one preferred embodiment of the provided method, the amino acid of formula 30 is prepared in one step by the reaction of an amine compound of formula 35, a boron compound of formula 36 or 37, and glyoxylic acid of formula 38 or its hydrated form:

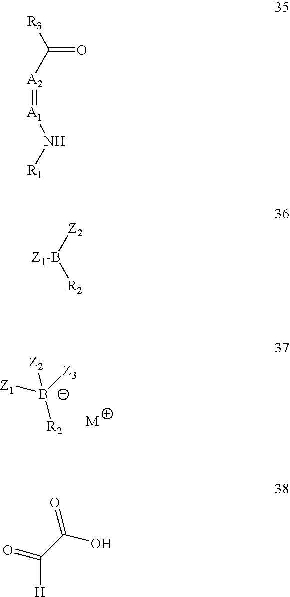

wherein:
R₁-R₃ and A₁-A₂ are defined as above;
Z₁-Z₃ are independently selected from the group consisting of hydroxy, alkoxy, acyloxy, fluoro, chloro, bromo, alkylamino, and arylamino; and
M is potassium or tetralkylamino.

In some preferred embodiments, the preparation of amino acid 30 using boron compounds 36 or 37 is performed according to U.S. Pat. No. 6,232,467, and the boron compound used is an organoboronic acid or boronate of formula 36, or a potassium trifluoroborate of formula 37.

The method of synthesis of N-heterocycles provided with the present invention is illustrated with the following applications to the synthesis of each of the provided compounds 2-29. In each case, the precursor amino acid 2A-29A is treated with an acid activator and a base to give directly 2-29:

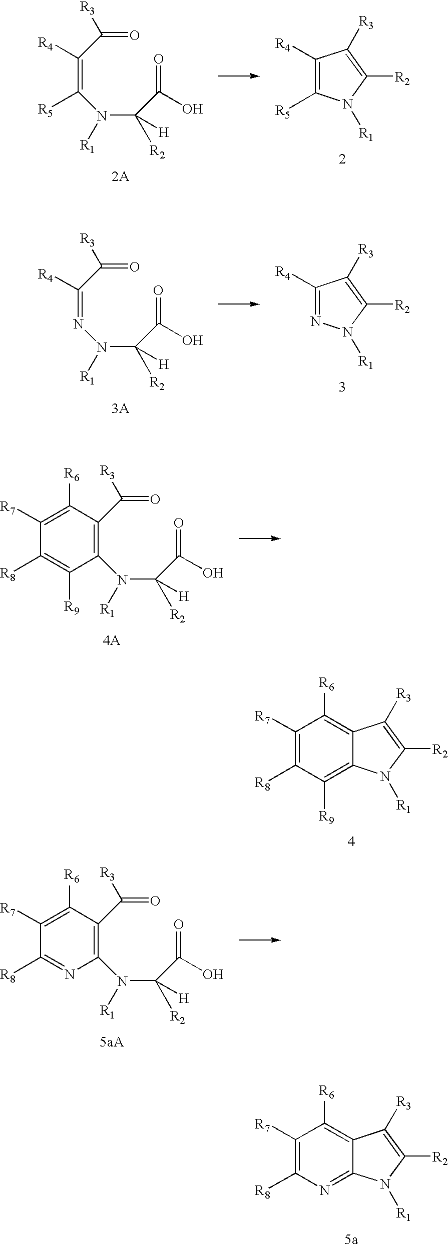

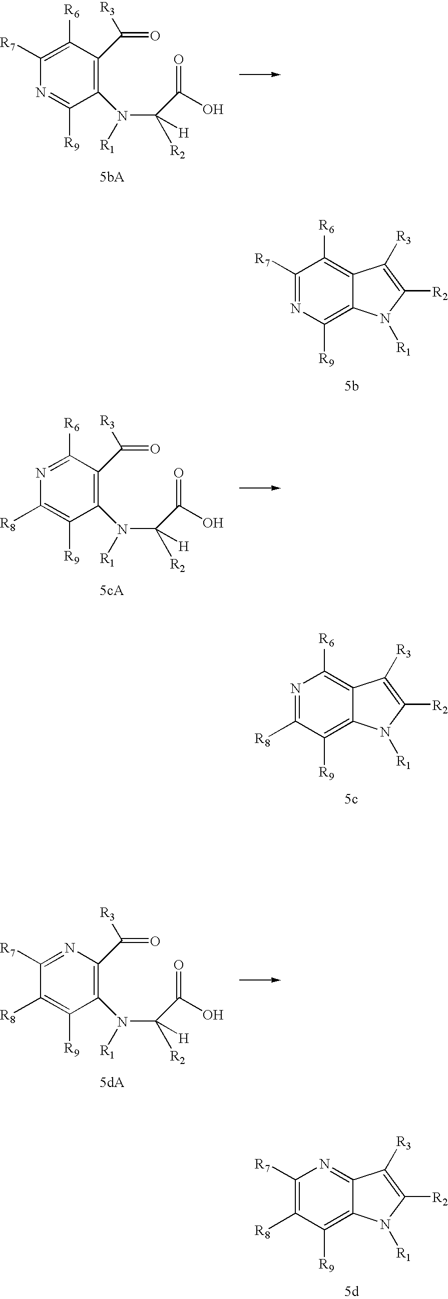

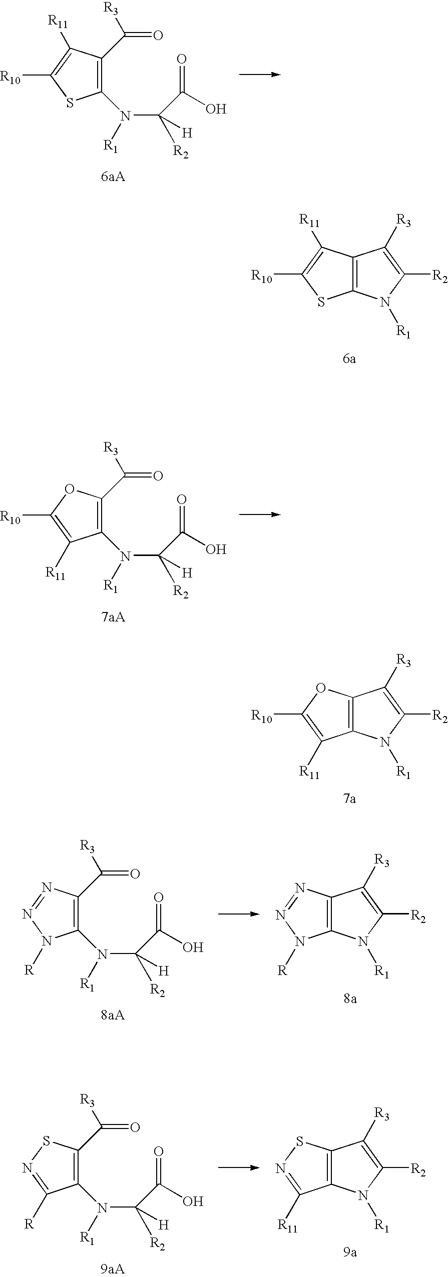

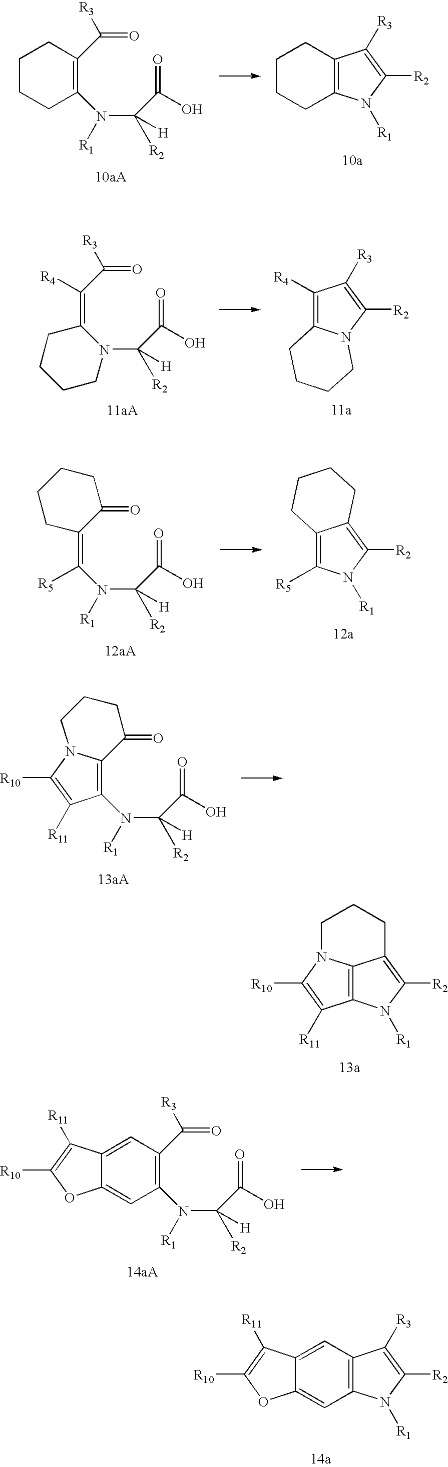

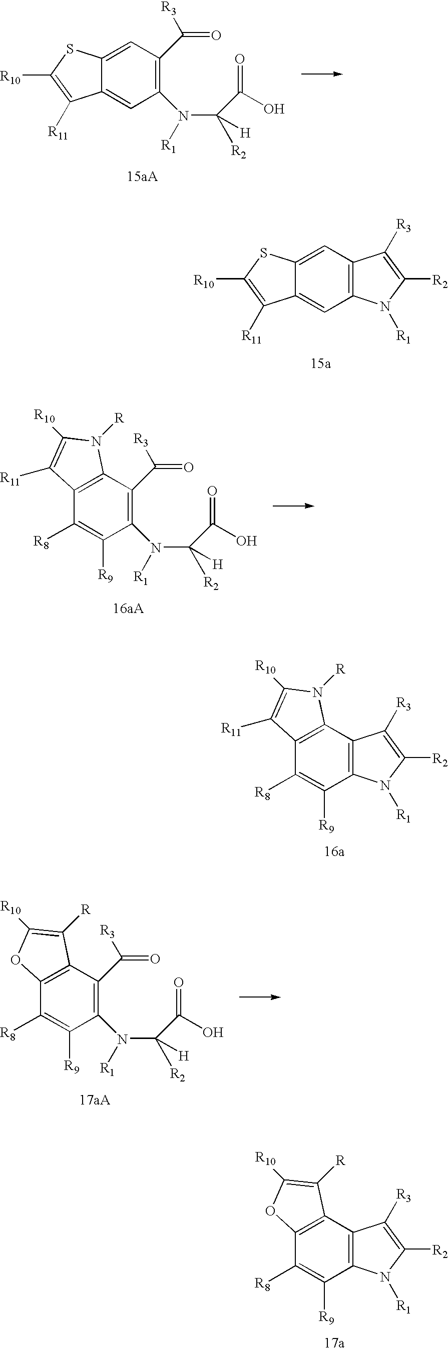

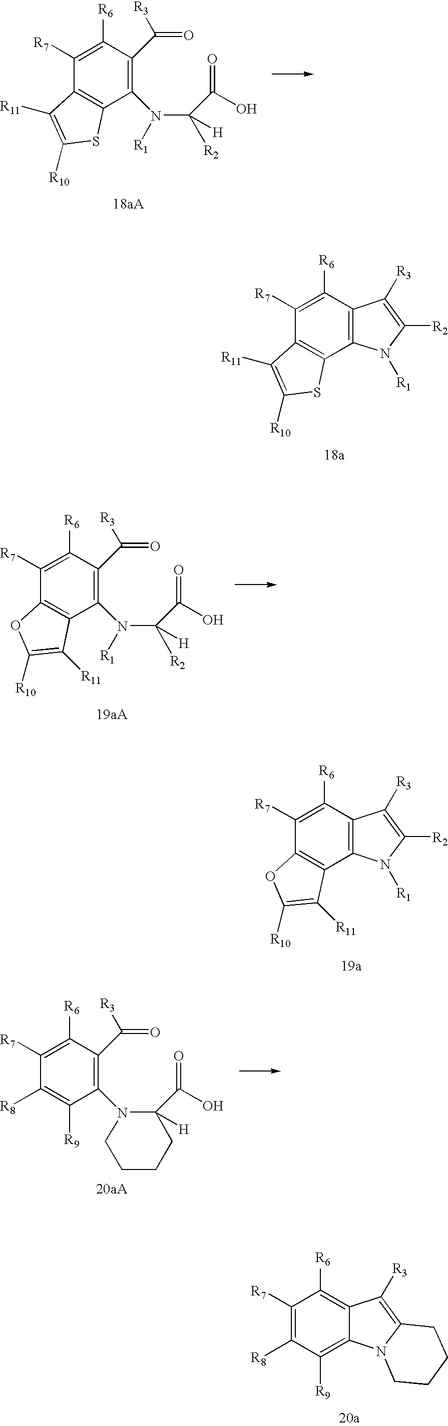

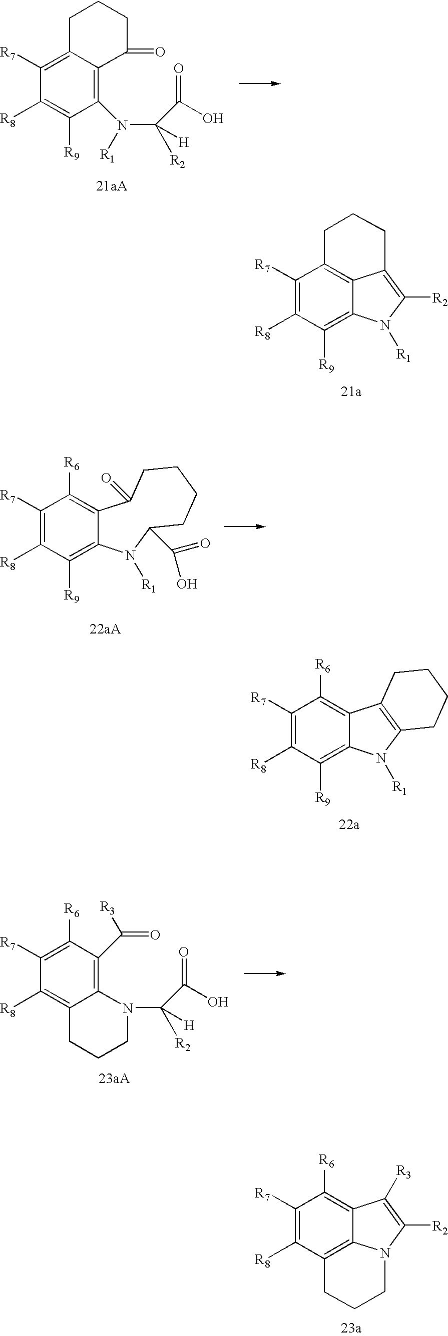

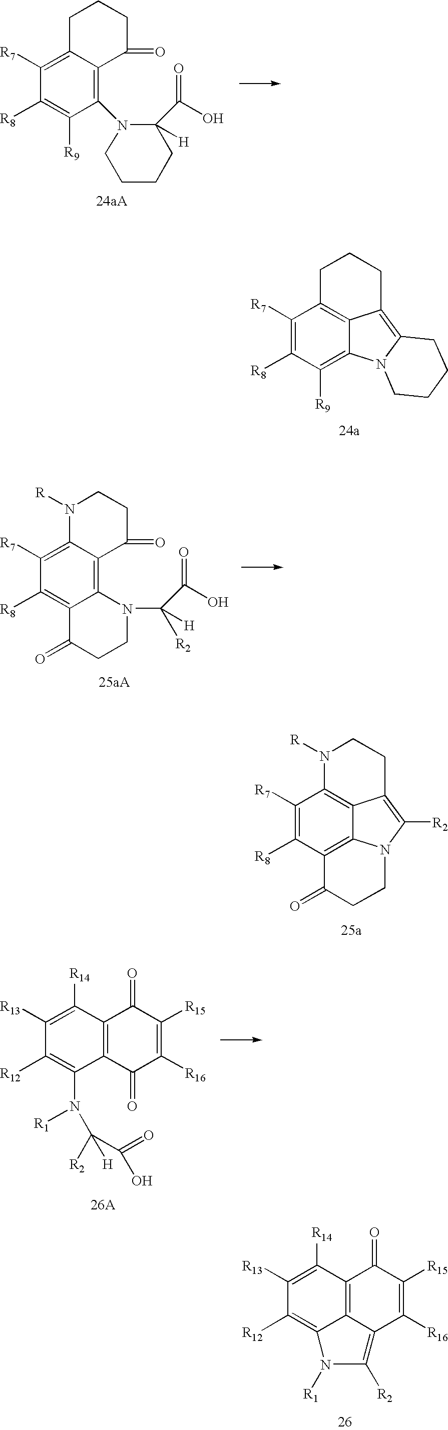

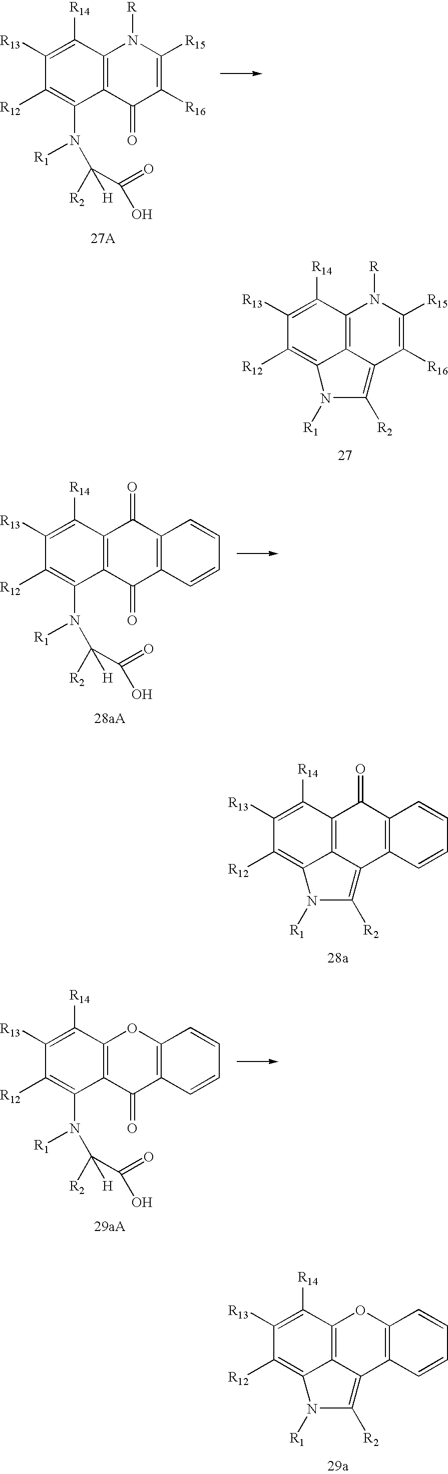

The required amine and amino acid precursors for the synthesis of the N-heterocycles provided with the present invention can be produced from commercially or readily available starting materials and by using methods known in the art, or by using new methods as described herein.

The following examples illustrate some of the benefits of the present invention in making available the provided novel compounds or in producing known N-heterocycles in a short, practical, efficient, and scalable manner. These examples show a wide range of methods to produce the key intermediates, followed by cyclization to form N-heterocycles according to the present invention. The provided examples are for the purpose of illustration and not intended to limit the scope of the invention.

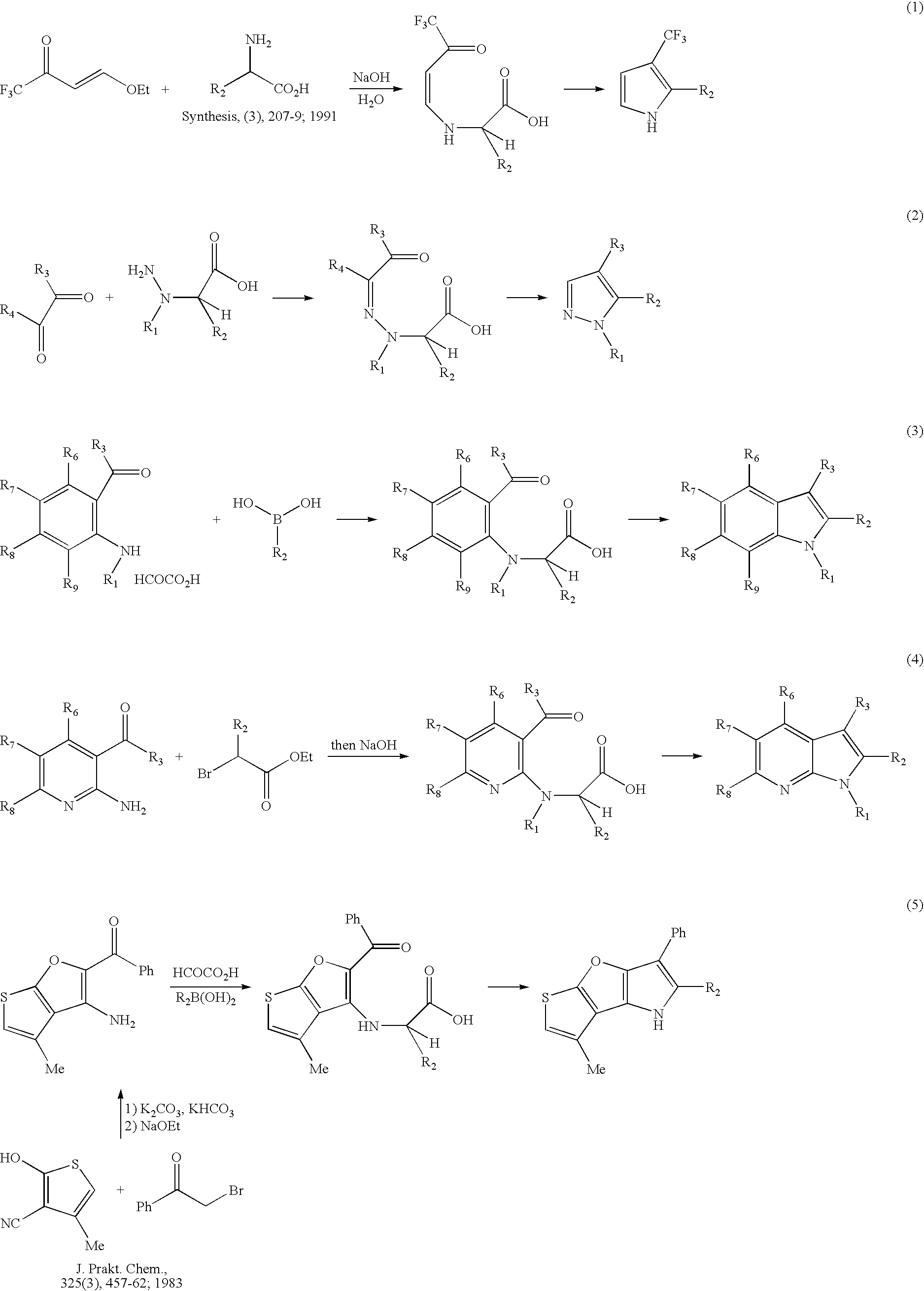

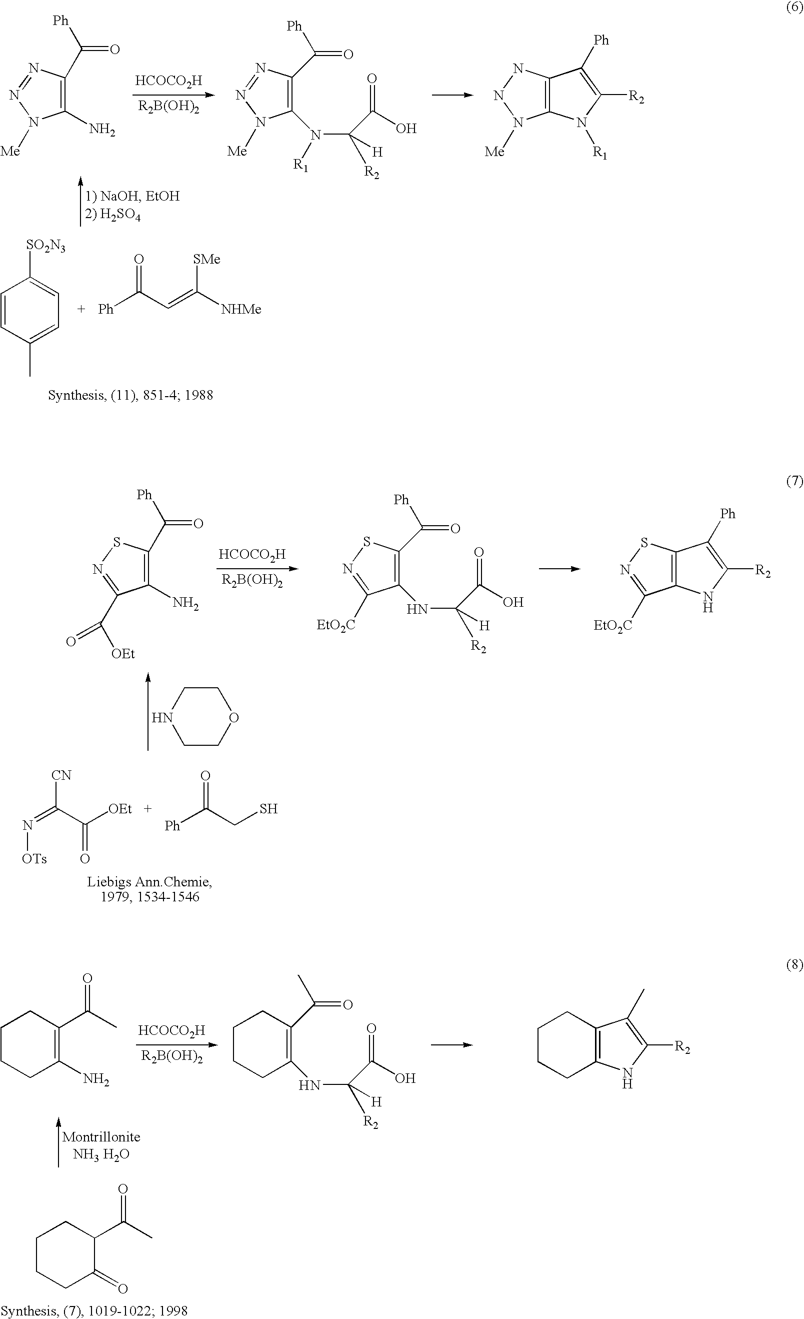

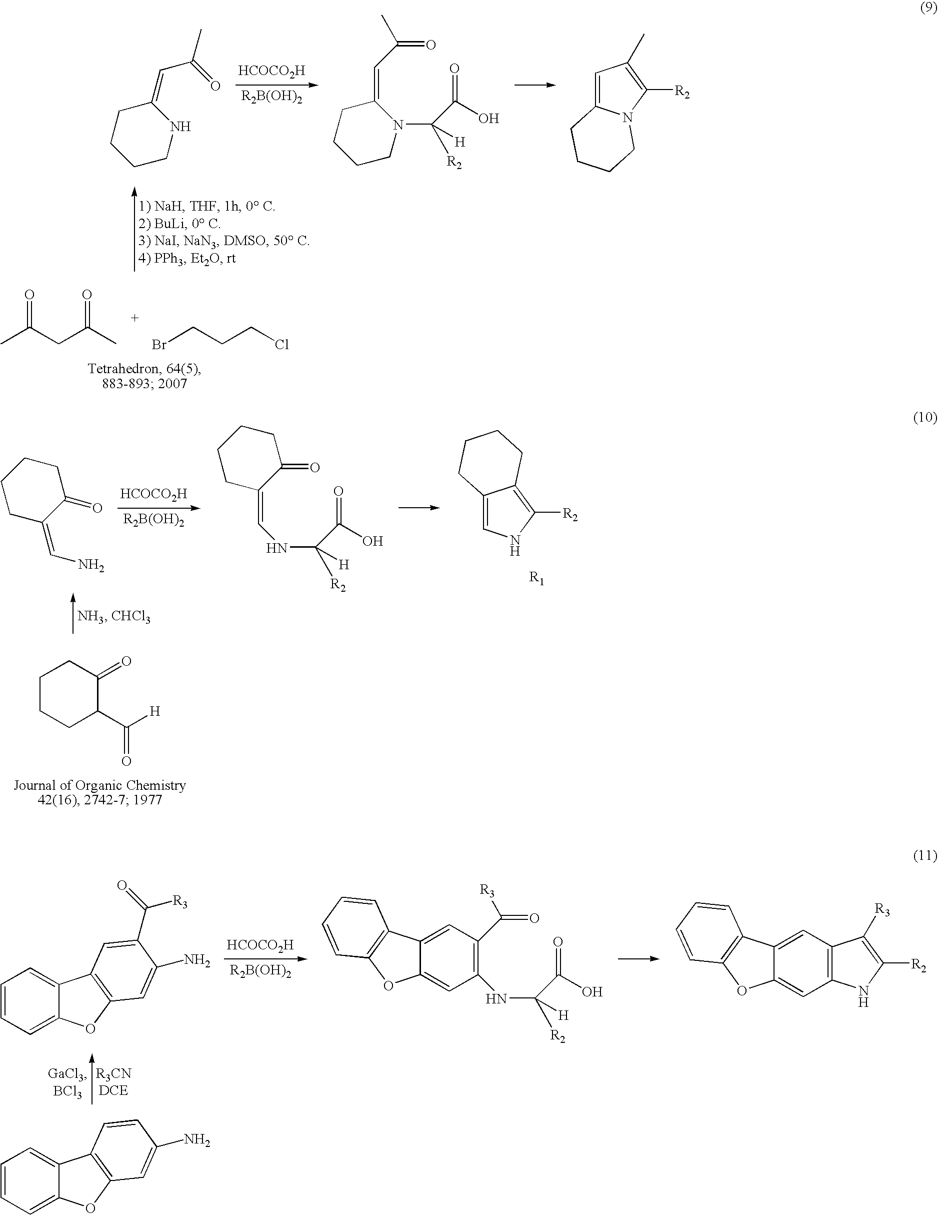

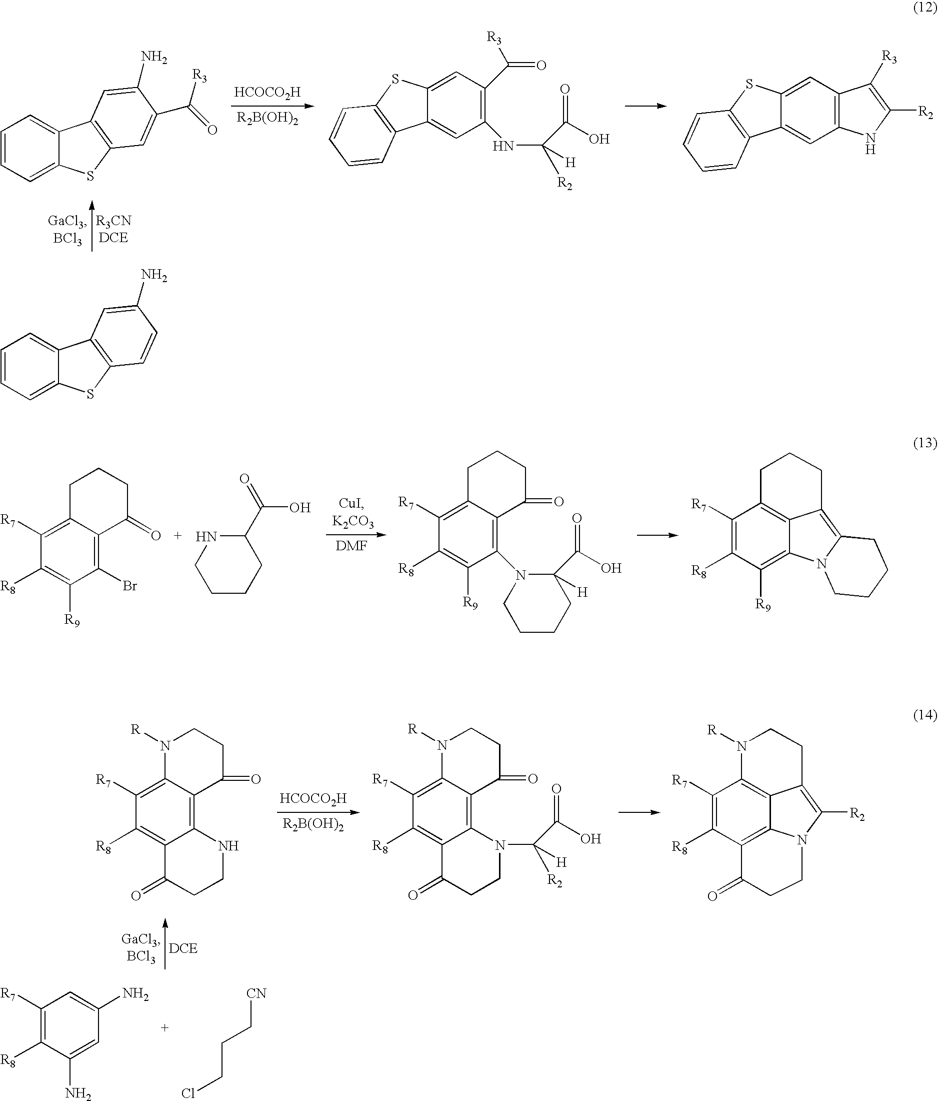

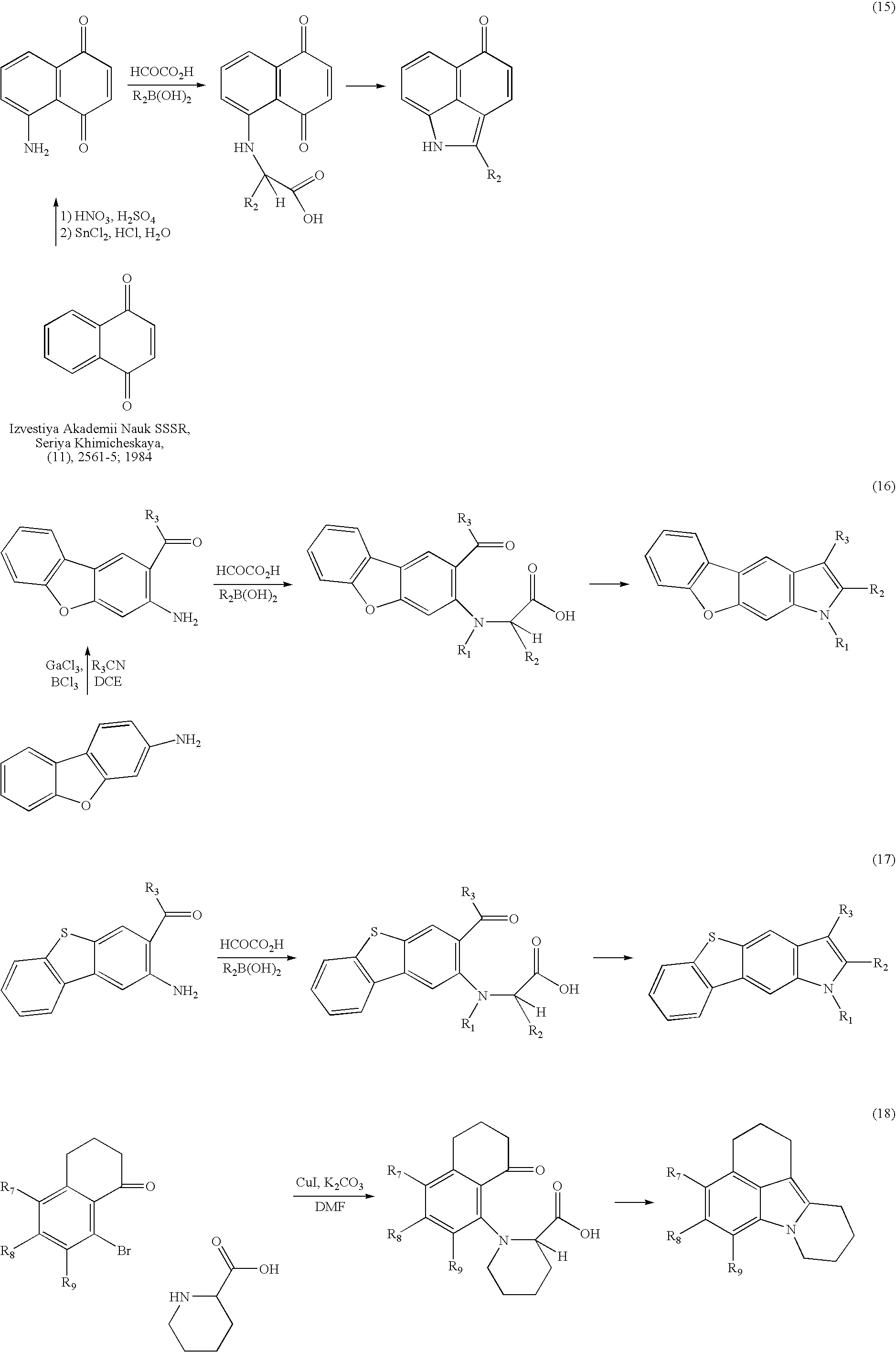

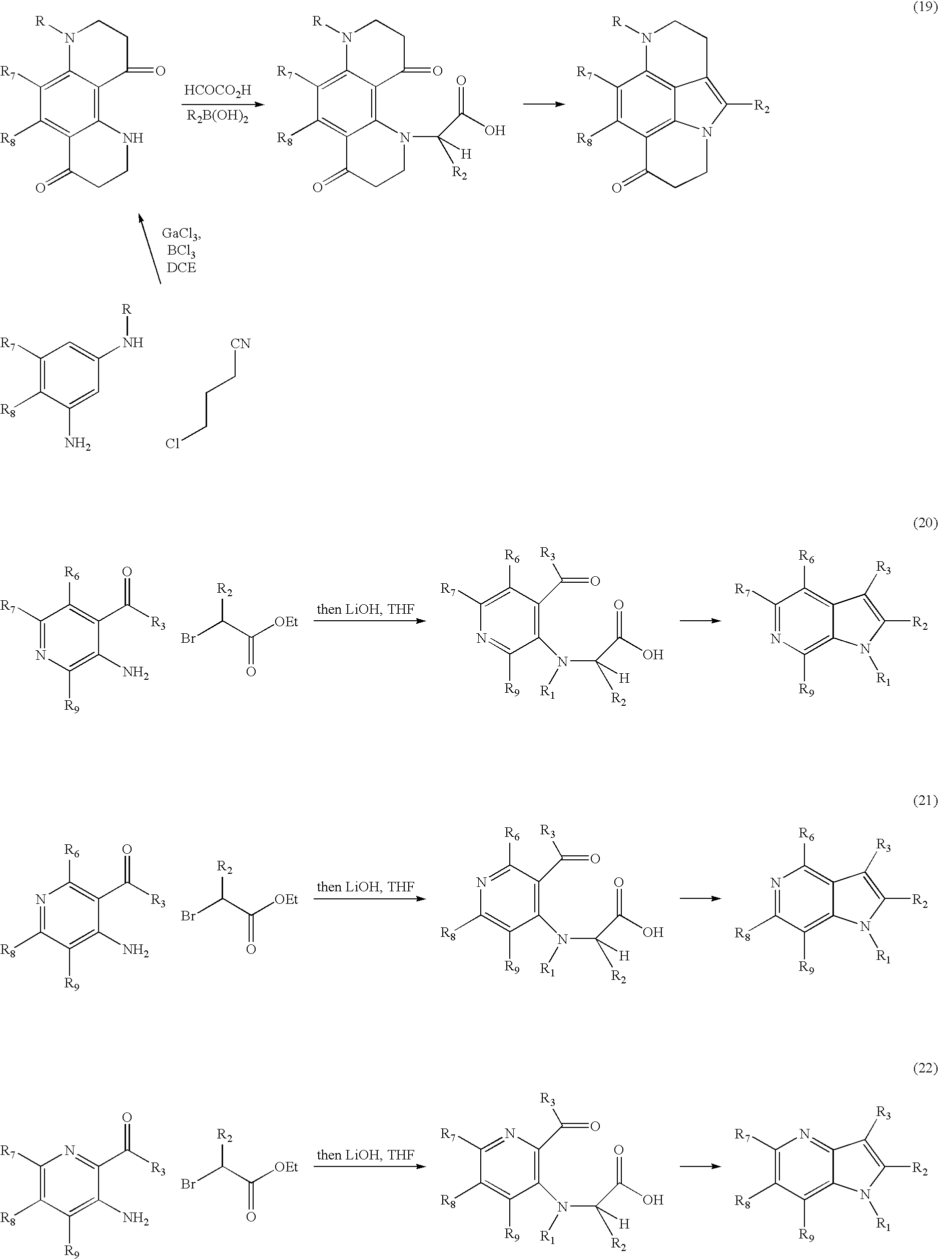

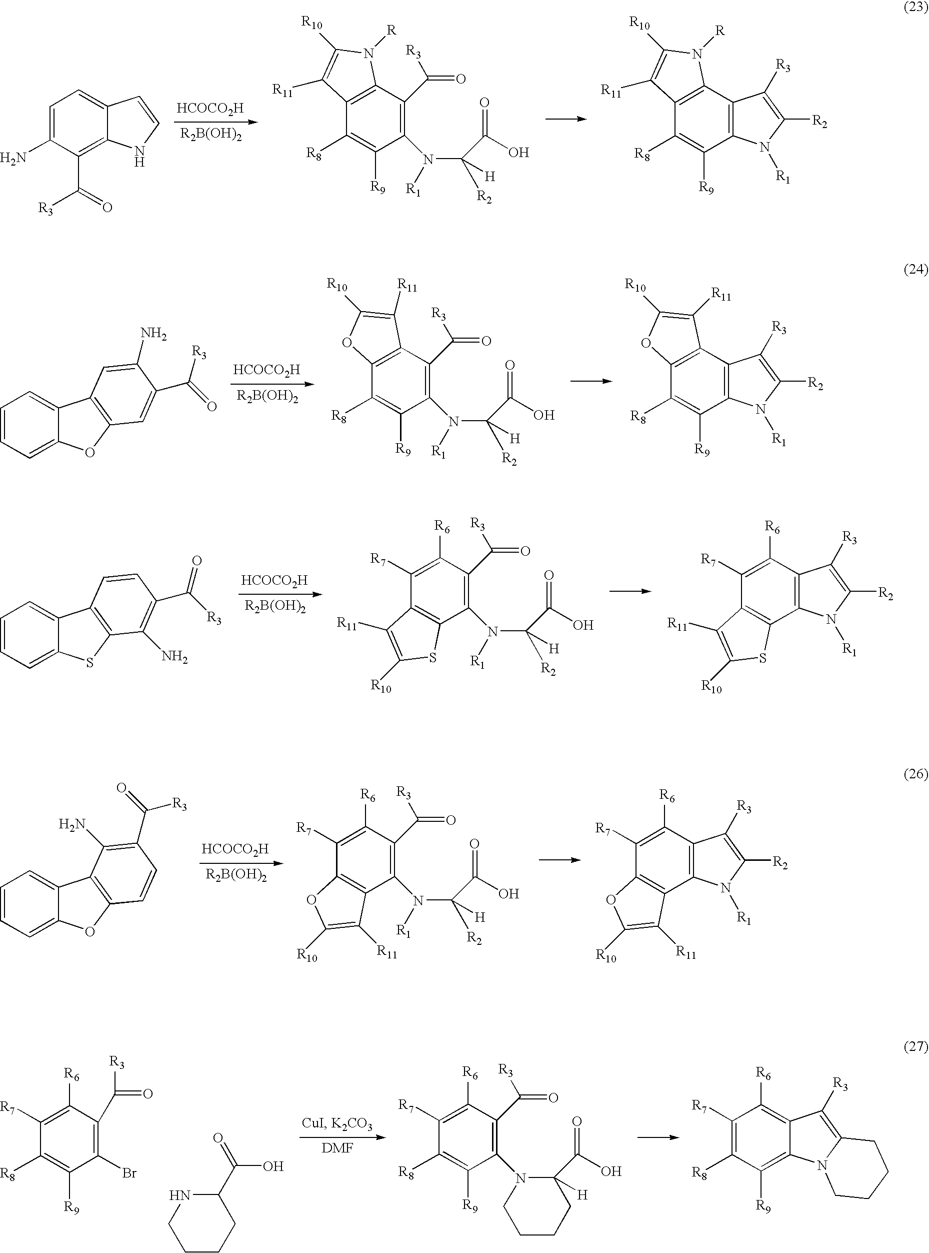

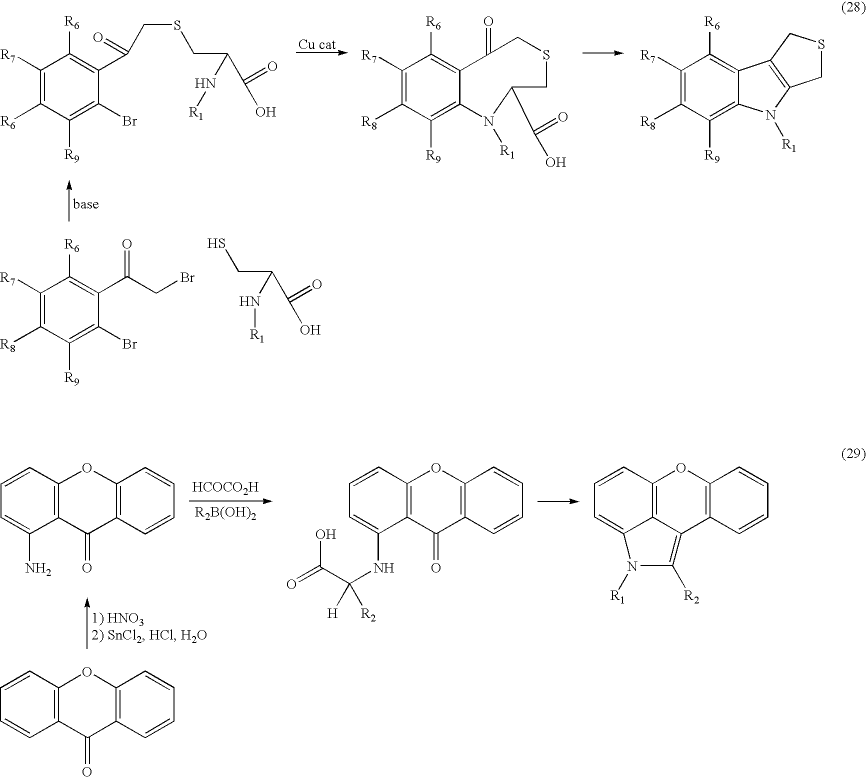

The method of synthesis provided by the present invention allows the efficient and concise synthesis of many valuable N-heterocycles that are used as pharmaceuticals, agrochemicals, or as chemical intermediates and chemical building blocks for the synthesis of novel materials.

In one embodiment, the provided synthesis can be used for the short synthesis of fluvastatin (Lescol), a clinically used pharmaceutical agent that contains an indole ring system. For the synthesis of fluvastatin, aniline F1 is reacted in Sugasawa reaction with a nitrile F2 to give amino ketone F3. One-step three-component reaction of F7 with boronic acid F4 and glyoxylic acid gives intermediate F5 that can be converted directly to fluvastatin using the method provided herein, followed by hydrolysis. Alternatively, using boron derivative F6 under similar conditions leads to compound F8, a known intermediate for the synthesis of fluvastatin.

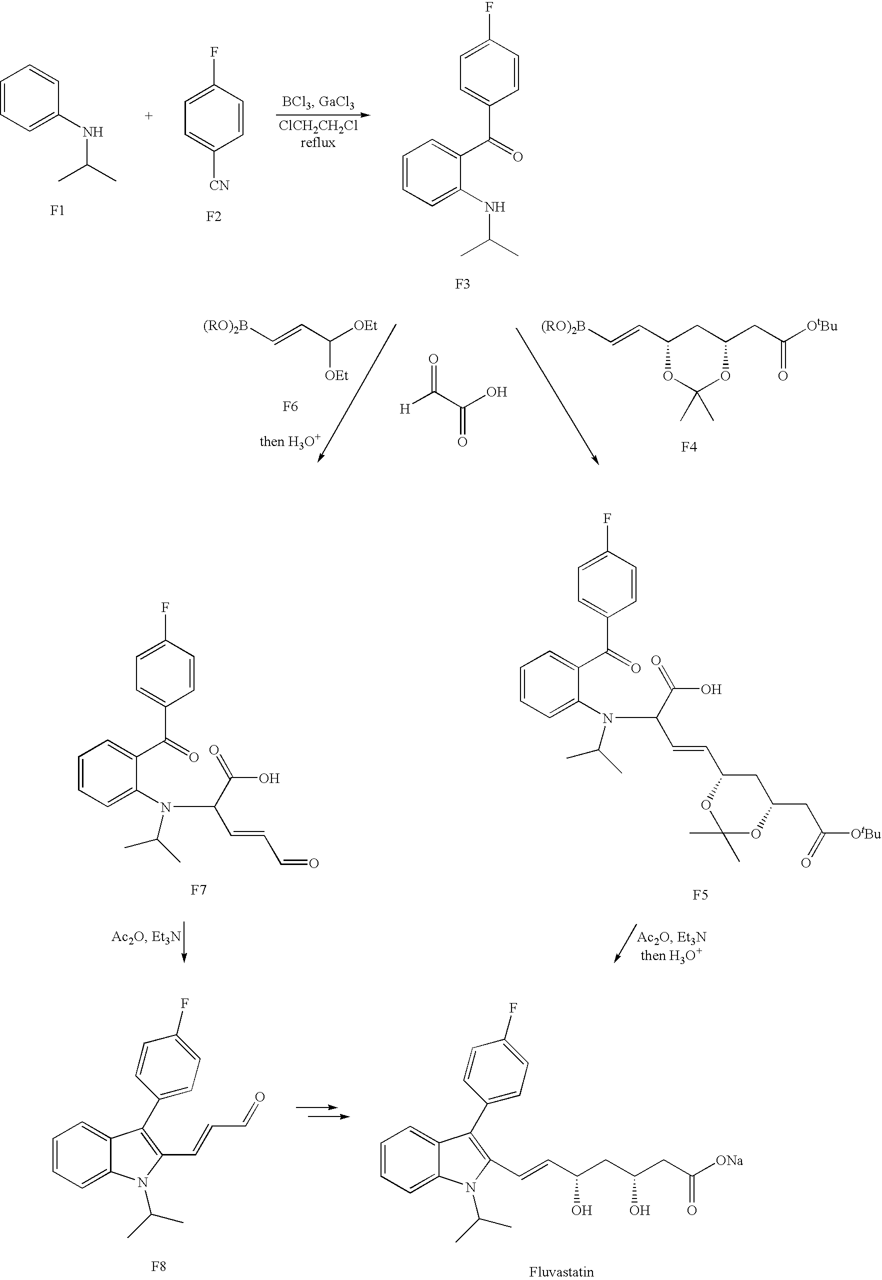

The compounds provided by the present invention exhibit potent activities against important biological targets and can be used as therapeutic agents against a number of diseases, including cancer. For example, compound 1-[5-chloro-2-(4-methoxy-phenyl)-3-trifluoromethyl-indol-1-yl]-ethanone, prepared under Example 2 of the present application, has shown potential anticancer activity against several cancer cell lines. For example, at 10 micromolar (μM) concentration, it has exhibited 80.8% cytotoxicity against the MDA MB-435 cell line, as determined by an MTT assay. Modeling of this compound showed strong affinity to the active site of integrin alpha v beta 3 (αvβ₃), the vitronectin receptor that is expressed in activated endothelial cells, melanoma, and glioblastomas.

The positioning of the CF3 group in the above molecule was found to be important for its activity, illustrating the utility of the provided method of synthesis that enables the synthesis of such compounds in an efficient manner. The ability of the present invention to conveniently incorporate fluorine-containing substituents at positions around the provided N-heterocycles is an important feature that results in the generation of potentially biologically active molecules. The introduction of fluorine and other halogen atoms in the structures of pharmaceutical and agrochemical agents is of great value and a large number of approved drugs contain at least one such atom.

Accordingly, the invention features a method of inhibiting cancer cells. The method comprises contacting a cancer cell with 1-[5-chloro-2-(4-methoxy-phenyl)-3-trifluoromethyl-indol-1-yl]-ethanone, thereby inhibiting the growth of the cell. In some embodiments, the cancer cell is a melanoma or glioblastoma cell.

The invention also provides methods for treating various diseases and conditions by administering to a subject in need thereof an effective amount of a compound of the invention. The diseases and disorders to be treated include inflammation, malaria, diseases and disorders caused by fungi, bacteria, viruses, and protozoan such as mycosis, cancer, depression, seasonal affective disorder, premenstrual dysphoric disorder, hypertension, migraine, rheumatoid arthritis, osteoarthritis, sepsis, asthma, adult respiratory distress syndrome, cardiovascular disorders, Alzheimer's disease, liver disease, cirrhosis, hepatocelular carcinoma, chronic hepatitis diseases, and disorders related to selective serotonin reuptake, tryptophan, DNA topoisomerase I, cancer chemotherapy, kinases, and immunity, neurotransmitters, sPLA2, MCP-1, glycogen phosphorylase, platelet activating factor (PAF), adenosine receptors, tyrosine kinases, GnRH, tranquillizers, angiogenisis, hepatitis C virus, plasmodia, cytotoxin, DNA intercalators, MDM2, HIV integrase, the ligand-binding pocket of PDZ domains of NHERF1 multi-functional adaptor protein, or neuroprotection.

“Subject,” as used herein, refers to a human or animal, including all vertebrates, e.g., mammals, such as primates (particularly higher primates), sheep, dog, rodents (e.g., mouse or rat), guinea pig, goat, pig, cat, rabbit, cow; and non-mammals, such as chicken, amphibians, reptiles, etc. In a preferred embodiment, the subject is a human. In another embodiment, the subject is an animal.

A subject to be treated may be identified, e.g., using diagnostic methods known in the art, as being suffering from a disease or an abnormal condition. The subject may be identified in the judgment of a subject or a health care professional, and can be subjective (e.g., opinion) or objective (e.g., measurable by a test or diagnostic method).

The term “treating” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable by some test or marker) or subjective (i.e., subject gives an indication of or feels an effect). The treatment methods can be performed alone or in conjunction with other drugs and/or radiotherapy. See, e.g., U.S. Patent Application 20040224363.

In one in vivo approach, a therapeutic compound itself is administered to a subject. Generally, the compound will be suspended in a pharmaceutically-acceptable carrier and administered orally or by intravenous (i.v.) infusion, or injected or implanted subcutaneously, intramuscularly, intrathecally, intraperitoneally, intrarectally, intravaginally, intranasally, intragastrically, intratracheally, or intrapulmonarily. The dosage required depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by i.v. injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

Furthermore, the compounds of the invention can be incorporated into pharmaceutical compositions. Such compositions typically include the compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration. Other active compounds can also be incorporated into the compositions.

A pharmaceutical composition is often formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In one embodiment, the compounds are prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. “Dosage unit form,” as used herein, refers to physically discrete units suited as unitary dosages for the subject to be treated; each unit containing a predetermined quantity of active compound calculated to produce the desired therapeutic effect in association with the required pharmaceutical carrier.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Example 1

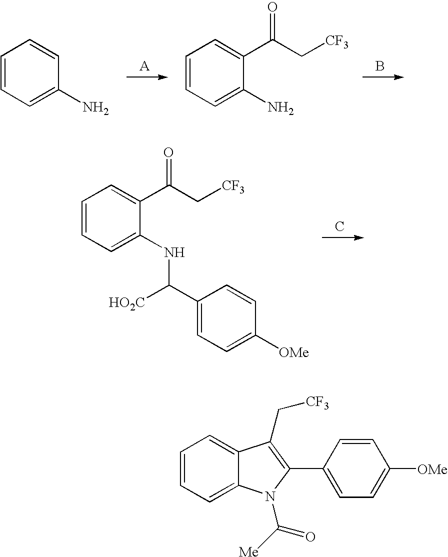

### Synthesis of 1-[2-(4-methoxy-phenyl)-3-(2,2,2-trifluoro-ethyl)-indol-1-yl]-ethanone

Step A: To a stirred solution of BCl₃ (10 mmol, 10 ml, 1 M) in dichloroethane at 0° C. was added aniline (10 mmol, 0.91 ml), followed by addition of 3,3,3-trifluoropropionitrile (10 mmol, 0.852 ml) and gallium trichloride (10 mmol, 1.76 g). The reaction mixture was warmed up to room temperature for about 30 minutes and refluxed for another 18 hours. After cooling, 1 N solution of hydrochloric acid was added, and the reaction was refluxed for an additional hour. The reaction mixture was neutralized with base and extracted with dichloromethane. The organic layer was evaporated under reduced pressure, and the residue was purified via flash chromatography in order to isolate 1-(2-amino-phenyl)-3,3,3-trifluoro-propan-1-one in good yield (1.12 g, 55% yield). ¹H NMR (400 MHz, CDCl₃): δ7.57 (d, J=8.2 Hz, 1H), 7.34 (t, J=7.0 Hz, 1H), 6.69 (t, J=8.4 Hz, 2H), 6.40 (broad s, 2H), 3.79 (q, J=10.3 Hz, 2H). ¹⁹F NMR (62.5 MHz, CDCl₃): δ-62.0. ¹³C NMR (100 MHz, CDCl₃): δ191.4, 151.2, 135.4, 130.9, 128.6 (q), 117.6, 116.8, 116.0, 42.5 (q).

Step B: The product of Step A (1 mmol) and glyoxylic acid monohydrate (1 mmol, 92 mg) were dissolved in 2 ml of acetonitrile. 1 mmol of p-methoxyphenyl boronic acid was added. The resulting reaction mixture was stirred at room temperature till TLC indicated that starting materials disappeared. The reaction mixture was concentrated under reduced pressure and the residue was purified via flash chromatography to afford (4-methoxy-phenyl)-[2-(3,3,3-trifluoro-propionyl)-phenylamino]-acetic acid in excellent yield (94%). ¹H NMR (400 MHz, acetone-d₆): δ9.92 (broad s, 1H), 7.91 (d, J=8.3 Hz, 1H), 7.5 (d, J=7.9 Hz, 2H), 7.36 (t, J=7.2 Hz, 1H), 6.98 (d, J=9.2 Hz, 2H), 6.69 (m, 2H), 5.82 (broad s, 1H), 5.37 (d, J=6.3 Hz, 1H), 4.21 (q, J_{H—F}=11.3 Hz, 2H), 3.82 (s, 3H). ¹⁹F NMR (376 MHz, acetone-d₆): δ-62.6. ¹³C NMR (62.5 MHz, acetone-d₆): δ193.3, 172.5, 160.8, 150.3, 136.4, 133.2, 130.6, 129.4, 126.1 (q, J_{C—F}=278.5 Hz), 116.1, 115.2, 114.1, 59.7, 55.6, 43.0 (q, J_{C—F}=27.3 Hz).

Step C: In a 1 dram vial acetic anhydride (1 ml) as a solvent, triethylamine (0.5 ml) and amino acid product of Step B (0.3 mmol) were mixed together. The reaction mixture was heated up to 90° C. and let stirred at this temperature for 30 minutes. After the reaction was completed (no amino acid on the TLC plate is left), the solvent was evaporated under the reduced pressure. The residue was purified by flash chromatography to yield 1-[2-(4-methoxy-phenyl)-3-(2,2,2-trifluoro-ethyl)-indol-1-yl]-ethanone in moderate yield (50%). ¹H NMR (400 MHz, CDCl₃): δ8.48 (d, J=8.1 Hz, 1H), 7.63 (d, J=7.5 Hz, 1H), 7.46-7.35 (m, 4H), 7.07 (d, J=8.5 Hz, 2H), 3.93 (s, 3H), 3.34 (q, J=10.5 Hz, 2H), 2.00 (s, 3H). ¹⁹F NMR (376 MHz, CDCl₃): δ-64.1. ¹³C NMR (100 MHz, CDCl₃): δ171.2, 160.4, 138.6, 136.6, 131.7, 128.8, 127.4, 125.6, 124.6, 124.0, 123.9, 121.9, 119.0, 116.5, 114.4, 111.2, 55.4, 30.3 (q, J=31.2 Hz), 27.6.

### Example 2

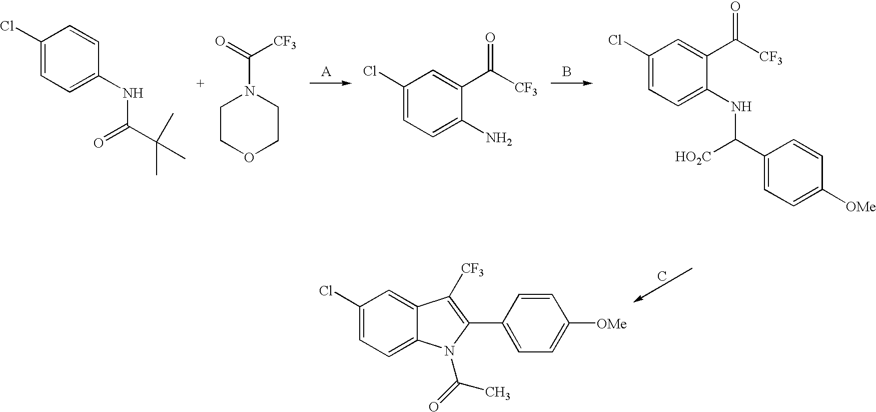

### Synthesis of 1-[5-chloro-2-(4-methoxy-phenyl)-3-trifluoromethyl-indol-1-yl]-ethanone

Step A1: To a solution of morpholine (30 mmol, 2.62 ml) in diethyl ether, trifluoroacetic acid anhydride was added dropwise while the reaction flask was chilled in an ice bath. After 3 hours, the reaction mixture was diluted with ethyl acetate and extracted with 1 N HCl. The organic layers were washed with sodium carbonate and brine, dried over sodium sulfate. The volatiles were removed under reduced pressure. The residue was purified via flash chromatography to obtain 2,2,2-trifluoro-1-morpholin-4-yl-ethanone as a colorless liquid in good yield (2.15 g, 78%). ¹H NMR (400 MHz, CDCl₃): δ3.68 (m, 8H). ¹⁹F NMR (376 MHz, CDCl₃): δ-69.1. ¹³C NMR (100 MHz, CDCl₃): δ155.6 q, 116.6 (q, J_{C—F}=297.7 Hz), 66.4, 46.3, 43.5.

Step A2: To a biphasic solution of p-chloroaniline (15 mmol, 1.92 g) in dichloromethane and 10% aqueous solution of sodium carbonate, pivaloyl chloride (16.5 mmol, 2.03 ml) was added dropwise. The reaction mixture was stirred intensively for 30 min. The reaction progress was followed by TLC. After reaction was completed, organic layer was separated and volatiles removed under reduced pressure to obtain N-(4-chloro-phenyl)-2,2-dimethyl-propionamide as a white solid in excellent yield (3.1 g, 98% yield). ¹H NMR (400 MHz, CDCl₃): δ7.50 (m, 2H), 7.28 (m, 2H), 1.33 (s, 9H). ¹³C NMR (100 MHz, CDCl₃): δ176.7, 136.6, 129.1, 128.9, 121.3, 39.6, 27.6.

Step A3: To a solution of N-(4-chloro-phenyl)-2,2-dimethyl-propionamide (product of Step A2) (14.2 mmol, 3 g) in dry THF (15 ml) under argon atmosphere, 22 ml of a 1.6 M solution of n-butyl lithium in hexanes was added at −50° C. The reaction mixture was left standing for 2 hours at 0° C. during which time a white precipitate formed. The mixture was cooled to −40° C. and solution of 2,2,2-trifluoro-1-morpholin-4-yl-ethanone (product of Step A1) (17 mmol, 3.2 g) in 10 ml of THF was added dropwise. After stirring for 1 hour at this temperature, the reaction mixture was quenched with saturated aqueous solution of ammonium chloride. The mixture was extracted with dichloromethane (2×50 ml), the organic layer was dried over anhydrous sodium sulfate, and evaporated under reduced pressure. The crude residue was used for the next step without further purification. The residue was dissolved in dioxane and 80 ml of 3 N HCl was added. The solution was refluxed for 12 hours. After cooling to room temperature, the solution was treated with ammonia and 1 N solution of sodium hydroxide and extracted with DCM. The organic layer was dried over anhydrous sodium sulfate, evaporated to yield a crude product. Purification was performed on silica gel via flash chromatography to obtain 1-(2-amino-5-chloro-phenyl)-2,2,2-trifluoro-ethanone as a yellow solid in good yield (2.6 g, 82% yield). ¹H NMR (400 MHz, CDCl₃): δ7.72 (m, 1H), 7.35 (m, 1H), 6.71 (d, J=9.1 Hz, 1H), 6.51 (broad s, 2H). ¹⁹F NMR (376 MHz, CDCl₃): δ-69.8. ¹³C NMR (100 MHz, CDCl₃): δ180.3 (q), 151.5, 136.9, 130.1, 130.0, 120.9, 119.0, 116.7 (q, J_{C—F}=291.4 Hz), 111.4.

Step B: To a solution of the product of Step A3 (1 eq., 1 mmol) and glyoxylic acid monohydrate (1 eq., 1 mmol, 92 mg) in 2 ml of acetonitrile, (1 eq., 1 mmol) p-methoxyphenyl boronic acid was added. The resulting reaction mixture was stirred at room temperature till TLC indicated that starting materials disappeared. The resulting reaction mixture was concentrated under reduced pressure and the residue was purified via flash chromatography to afford [4-chloro-2-(2,2,2-trifluoro-acetyl)-phenylamino]-(4-methoxy-phenyl)-acetic acid in good yield (84%). ¹H NMR (400 MHz, methanol-d₄): δ7.72 (broad s, 1H), 7.45-7.38 (m, 3H), 7.06 (m, 1H), 6.96-6.92 (m, 2H), 6.73 (m, 1H), 6.35 (d, J=9.0 Hz, 1H), 5.29 (s, 1H), 5.02 (d, J=15.7 Hz, 1H), 3.80 (s, 3H). ¹⁹F NMR (376 MHz, methanol-d₄): δ-70.8. ¹³C NMR (250 MHz, methanol-d₄): δ181.0 (q, J_{C—F}=33.9 Hz), 174.9, 161.3, 151.3, 145.7, 138.2, 131.6, 131.4, 130.6, 129.5, 129.4, 121.4 (t, J_{C—F}=28.8 Hz), 117.1, 115.5, 114.9, 61.1, 55.8.

Step C: In a 1 dram vial acetic anhydride as a solvent, triethylamine (0.5 ml) and amino acid (0.3 mmol) were mixed together. The reaction mixture was heated till 90° C. and let stirred at this temperature for 30 minutes. After the reaction was completed (no amino acid on TLC plate is left) the solvent was evaporated under reduced pressure. The residue was purified by flash chromatography to yield 1-[5-chloro-2-(4-methoxy-phenyl)-3-trifluoromethyl-indol-1-yl]-ethanone in good yield (84%). ¹H NMR (400 MHz, CDCl₃): δ8.33 (d, J=8.9 Hz, 1H), 7.77 (s, 1H), 7.42-7.39 (m, 3H), 7.06 (d, J=9.3 Hz, 2H), 3.93 (s, 3H), 1.96 (s, 3H). ¹⁹F NMR (376 MHz, CDCl₃): δ-54.5. ¹³C NMR (62.5 MHz, CDCl₃): δ171.3, 161.1, 132.9 (q, J=352.3 Hz), 131.5, 126.2, 122.3, 119.2 (q), 117.3, 114.3, 55.4, 27.6.

### Example 3

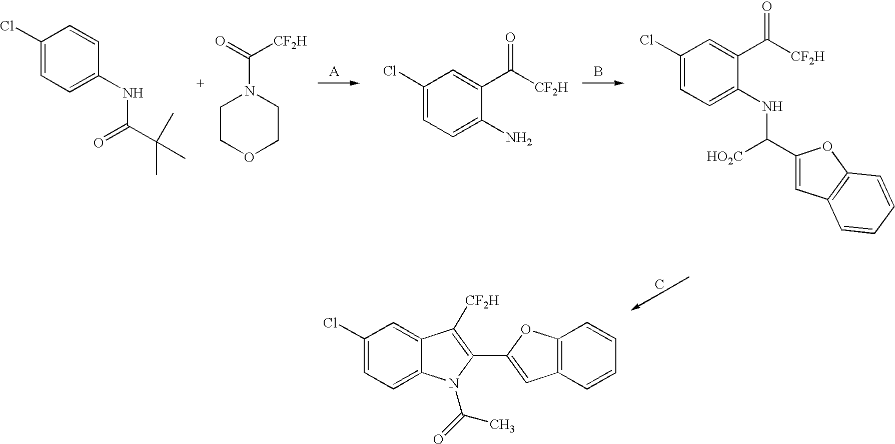

### Synthesis of 1-(2-benzofuran-2-yl-5-chloro-3-difluoromethyl-indol-1-yl)-ethanone

Step A1: It was prepared from morpholine and difluoroacetic anhydride in good yield following the procedure in Example 2 (4.1 g, 83%). ¹H NMR (400 MHz, CDCl₃): δ6.11 (t, J=53.4 Hz, 1H), 3.71 (m, 4H), 3.63 (m, 4H). ¹⁹F NMR (376 MHz, CDCl₃): δ-121.5 (d). ¹³C NMR (100 MHz, CDCl₃): δ160.6 (t, J_{C—F}=28.1 Hz), 110.5 (t, J_{C—F}=244.9 Hz), 66.5, 66.4, 45.3, 42.6.

Step A2: It was prepared from N-(4-chloro-phenyl)-2,2-dimethyl-propionamide (product of Example 2, Step B) and 2,2-difluoro-1-morpholin-4-yl-ethanone (the product of PART A) in good yield (59%). ¹H NMR (400 MHz, CDCl₃): δ7.79 (d, J=2 Hz, 1H), 7.32 (m, 1H), 6.70 (d, J=9.0 Hz, 1H), 6.45 (broad s, 2H), 6.31 (t, J_{H—F}=53.2 Hz, 1H). ¹⁹F NMR (376 MHz, CDCl₃): δ-120.6 (d). ¹³C NMR (100 MHz, CDCl₃): δ187.4 (t, J_{C—F}=35.5 Hz), 150.8, 136.3, 130.0, 129.9, 129.8, 120.6, 118.9, 113.6, 111.1 (t, J_{C—F}=256.1 Hz).

Step B: To a solution of the product of Step A2 (1 eq., 1 mmol) and glyoxylic acid monohydrate (1 eq., 1 mmol, 92 mg) in acetonitrile, benzofuran-2-boronic acid (1 eq., 1 mmol) was added. The resulting reaction mixture was stirred at room temperature till TLC indicated that starting materials disappeared. The resulting suspension was concentrated under reduced pressure and the residue was purified via flash chromatography to afford benzofuran-2-yl-[4-chloro-2-(2,2-difluoro-acetyl)-phenylamino]-acetic acid in good yield 75%. ¹H NMR (400 MHz, acetone-d₆): δ9.83 (d, J=5.5 Hz, 1H), 7.90 (s, 1H), 7.60-7.44 (m, 3H), 7.31-7.24 (m, 2H), 7.04 (m, 2H), 5.81 (s, 1H), 4.09 (q, J=6.8 Hz, 1H). ¹⁹F NMR (376 MHz, acetone-d₆): δ-124.9 (q, J=18.2 Hz). ¹³C NMR (62.5 MHz, acetone-d₆): δ188.0 (t, J=23 Hz), 170.2, 155.7, 153.8, 149.9, 137.0, 131.4, 128.9, 125.4, 123.9, 122.1, 120.6, 116.0, 115.3, 114.3, 112.0, 110.4, 106.6, 55.1.

Step C: In a 1 dram vial acetic anhydride as a solvent, triethylamine (0.5 ml) and amino acid product of Step B (0.3 mmol) were mixed together. The reaction mixture was heated up to 90° C. and let stirred at this temperature for 30 minutes. After the reaction was completed (no amino acid on TLC plate was left) the solvent was evaporated under reduced pressure. The residue was purified by flash chromatography to yield 1-(2-benzofuran-2-yl-5-chloro-3-difluoromethyl-indol-1-yl)-ethanone in moderate yield (42%). ¹H NMR (400 MHz, CDCl₃): δ8.30 (d, J=9.1 Hz, 1H), 7.85 (s, 1H), 7.65 (d, J=7.7 Hz, 1H), 7.51 (d, J=8.2 Hz, 1H), 7.38 (m, 2H), 7.31 (t, J=7.0 Hz, 1H), 7.08 (s, 1H), 6.58 (t, J_{H—F}=54.2 Hz, 1H), 2.14 (s, 3H). ¹⁹F NMR (376 MHz, CDCl₃): δ-108.7 (d, J=53.4 Hz). ¹³C NMR (62.5 MHz, CDCl₃): δ170.4, 155.7, 143.4, 130.3, 127.5, 126.6, 124.2, 122.1, 120.6, 117.6, 115.9, 112.2, 111.8, 111.3, 108.5, 25.1.

### Example 4

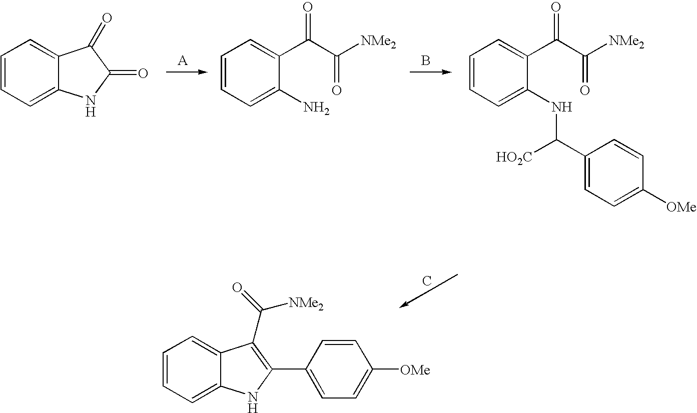

### Synthesis of 2-(4-methoxy-phenyl)-1H-indole-3-carboxylic acid dimethylamide

Step A: A solution of isatin (50 mmol, 7.36 g) and aqueous dimethylamine (40%, 40 ml) was refluxed for 10 min. The reaction was allowed to cool down to room temperature. The precipitated yellow solid was collected by filtration to afford 2-(2-amino-phenyl)-N,N-dimethyl-2-oxo-acetamide in moderate yield (6.15 g, 64%). ¹H NMR (250 MHz, CDCl₃): δ7.36 (d, J=9.4 Hz, 1H), 7.29-7.21 (m, 1H), 6.65-6.55 (m, 2H), 3.05 (s, 3H, Me), 2.91 (s, 3H, Me). ¹³C NMR (62.5 MHz, CDCl₃): δ194.2, 167.4, 151.6, 135.8, 133.0, 117.0, 116.2, 114.0, 37.1, 33.8.

Step B: To a solution of amine product of Step A (1 eq.) and glyoxylic acid monohydrate (1 eq.) in acetonitrile, 1 eq. of p-methoxyphenyl boronic acid was added. The resulting reaction mixture was stirred at room temperature till TLC indicated that starting materials disappeared. The resulting suspension was concentrated under reduced pressure and the residue was purified via flash chromatography to afford (2-dimethylaminooxalyl-phenylamino)-(4-methoxy-phenyl)-acetic acid in good yield (65%). ¹H NMR (250 MHz, methanol-d₄): δ7.39 (d, J=8.6 Hz, 2H), 7.28 (t, J=7.7 Hz, 1H), 6.89 (d, J=8.5 Hz, 2H), 6.64-6.58 (m, 2H), 5.24 (s, 1H), 3.72 (s, 3H), 3.05 (s, 3H), 2.92 (s, 3H). ¹³C NMR (62.5 MHz, methanol-d₄): δ195.3, 174.0, 169.2, 161.1, 151.2, 137.7, 134.8, 130.8, 129.4, 116.9, 115.3, 114.4, 60.2, 55.7, 37.5, 34.1.

Step C: To a suspension of the amino acid product of Step B (1 eq., 0.3 mmol) in toluene, p-toluenesulfonic acid chloride was added (1 eq., 0.3 mmol), followed by addition of triethylamine (2 eq., 0.6 mmol). The reaction mixture was stirred at room temperature for 4 hours and extracted with water and ethyl acetate. The organic residue was dried with sodium sulfate then purified on silica gel (10% ethyl acetate:hexane) to afford 2-(4-methoxy-phenyl)-1H-indole-3-carboxylic acid dimethylamide in good yield (55%). ¹H NMR (400 MHz, CDCl₃): δ9.07 (s, 1H), 7.56 (d, J=9.0 Hz, 1H), 7.36 (d, J=9.0 Hz, 2H), 7.13 (m, 2H), 6.76 (d, J=8.7 Hz, 2H), 3.78 (s, 3H), 3.14 (s, 3H), 2.74 (s, 3H). ¹³C NMR (100 MHz, methanol-d₄): δ170.1, 160.2, 136.8, 136.1, 128.4, 127.2, 124.4, 122.1, 120.1, 118.6, 114.0, 110.8, 54.3, 37.6, 33.9.

### Example 5

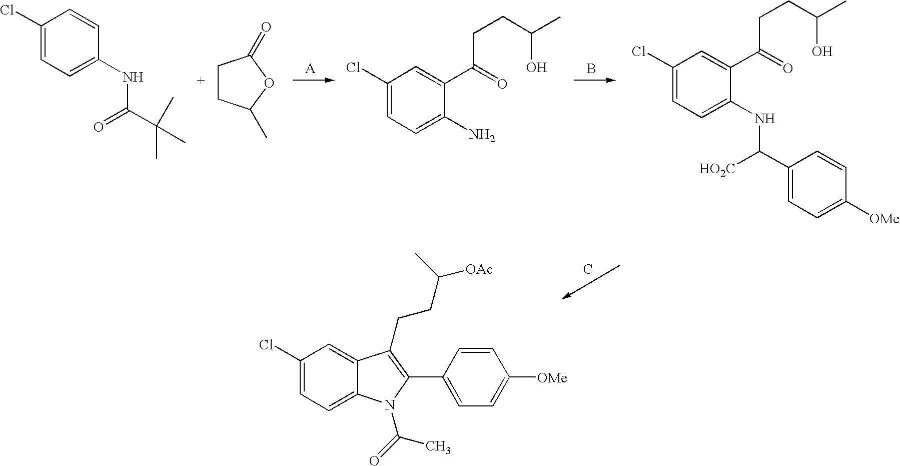

### Synthesis of acetic acid 3-[1-acetyl-5-chloro-2-(4-methoxy-phenyl)-1H-indol-3-yl]-1-methyl-propyl ester

Step A: To a solution of N-(4-chloro-phenyl)-2,2-dimethyl-propionamide (14.2 mmol, 3 g) in dry THF (15 ml) under argon atmosphere 22 ml of a 1.6 M solution of n-butyl lithium in hexanes was added at −50° C. The reaction mixture was stood for 2 hours at 0° C. during which time a white precipitate formed. The mixture was cooled to −40° C. and solution of γ-valerolactone (17 mmol, 1.6 ml) in 10 ml of dry THF was added dropwise. After stirring for 1 hour at this temperature, the reaction mixture was quenched with saturated aqueous solution of ammonium chloride. The mixture was extracted with dichloromethane (2×50 ml), the organic layer was dried over anhydrous sodium sulfate, and evaporated under reduced pressure. The crude residue was used for the next step without further purification. The residue was dissolved in dioxane and 80 ml of 3 N HCl was added. The solution was refluxed for 12 hours. After cooling to room temperature, the solution was neutralized with 1 N solution of sodium hydroxide followed by extraction with DCM. The organic layer was dried over anhydrous sodium sulfate, evaporated to yield a crude product. Purification was performed on silica gel (ethyl acetate:hexanes) to obtain 1-(2-amino-5-chloro-phenyl)-4-hydroxy-pentan-1-one as a yellow solid in moderate yield (0.96 g, 29% yield). ¹H NMR (250 MHz, CDCl₃): δ7.73 (d, J=2.4 Hz, 1H), 7.18 (dd, J=8.8 Hz, 1H), 6.60 (d, J=8.7 Hz, 1H), 6.25 (broad s, 2H), 3.92-3.81 (m, 1H), 3.10-3.04 (m, 2H), 1.89-1.81 (m, 2H), 1.24 (d, J=6.2 Hz, 3H). ¹³C NMR (250 MHz, CDCl₃): δ202.0, 148.8, 134.3, 130.3, 120.0, 118.8, 67.5, 35.5, 33.3, 23.8.

Step B: To a solution of the product of Step A (1 eq., 1 mmol) and glyoxylic acid monohydrate (1 eq., 1 mmol, 92 mg) in acetonitrile, p-methoxyphenyl boronic acid (1 eq., 1 mmol) was added. The resulting reaction mixture was stirred at room temperature till TLC indicated that starting materials disappeared. The resulting mixture was concentrated under reduced pressure and the residue was purified via flash chromatography to afford the desired amino acid which is crude for Step C.

Step C: To a suspension of the amino acid product of Step B (1 eq., 0.3 mmol) in toluene, p-toluenesulfonic acid chloride was added (1 eq., 0.3 mmol), followed by addition of triethylamine (2 eq., 0.6 mmol). The reaction mixture was stirred at room temperature for 4 hours and extracted with water and ethyl acetate. The organic residue was dried with sodium sulfate then purified on silica gel (ethyl acetate:hexane) to afford acetic acid 3-[1-acetyl-5-chloro-2-(4-methoxy-phenyl)-1H-indol-3-yl]-1-methyl-propyl ester in moderate yield (39%). ¹H NMR (400 MHz, CDCl₃): δ8.40 (d, J=9.0 Hz, 1H), 7.50 (s, 1H), 7.35-7.30 (m, 3H), 7.05 (d, J=8.5 Hz, 2H), 4.84 (m, 1H), 3.92 (s, 3H), 2.54 (m, 2H), 1.99 (s, 3H), 1.97 (s, 3H), 1.89-1.72 (m, 2H), 1.20 (d, J=6.2 Hz, 3H). ¹³C NMR (100 MHz, CDCl₃): δ171.0, 170.8, 160.1, 136.3, 135.2, 131.5, 130.6, 128.9, 125.1, 124.7, 120.8, 118.1, 117.8, 114.3, 70.4, 55.3, 36.0, 27.5, 21.3, 20.2, 19.7.

### Example 6

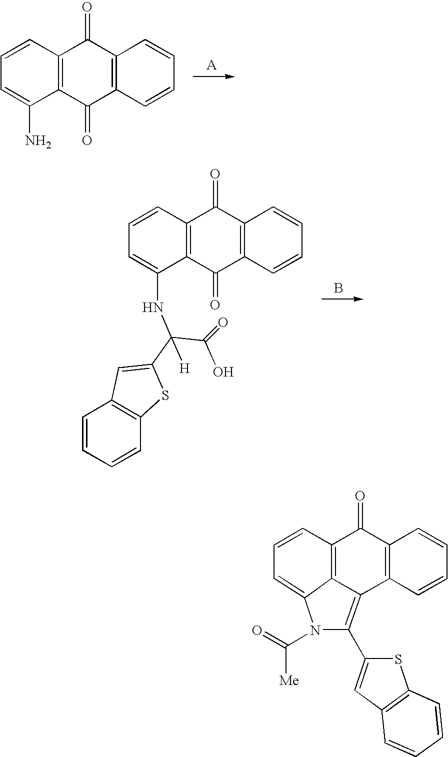

### Synthesis of 2-acetyl-1-benzo[b]thiophen-2-yl-2H-2-aza-aceanthrylen-6-one

Step A: To a solution of commercially available amine (1 eq., 1 mmol) and glyoxylic acid monohydrate (1 eq., 1 mmol, 92 mg) in acetonitrile, benzo[b]thiophene-2-boronic acid (1 mmol, 1 eq.) was added. The resulting reaction mixture was stirred at room temperature till TLC indicated that starting materials disappeared. The resulting reaction mixture was concentrated under reduced pressure to afford the crude amino acid.

Step B: In a 1 dram vial acetic anhydride as a solvent, triethylamine (0.5 ml) and amino acid from Step A (0.3 mmol) were mixed together. The reaction mixture was heated up to 90° C. and let stirred at this temperature for 30 minutes. After the reaction was completed (no amino acid on TLC plate was left), the solvent was evaporated under reduced pressure. The residue was purified by flash chromatography to yield 2-acetyl-1-benzo[b]thiophen-2-yl-2H-2-aza-aceanthrylen-6-one in moderate yield (33%). ¹H NMR (400 MHz, CDCl₃): δ8.67 (d, J=7.6 Hz, 1H), 8.54 (d, J=8.8 Hz, 1H), 8.29 (d, J=7.6 Hz, 1H), 8.06-8.00 (m, 2H), 7.72 (t, J=8.8 Hz, 2H), 7.60-7.57 (m, 2H), 7.48 (t, J=7.2 Hz, 1H), 7.41-7.35 (m, 2H), 2.35 (s, 3H). ¹³C NMR (100 MHz, CDCl₃): δ183.3, 170.8, 141.5, 139.2, 135.7, 133.0, 132.9, 132.7, 131.8, 129.0, 128.7, 128.0, 127.6, 126.1, 125.4, 124.8, 124.2, 122.9, 122.8, 122.2, 116.6, 26.1.

### Example 7

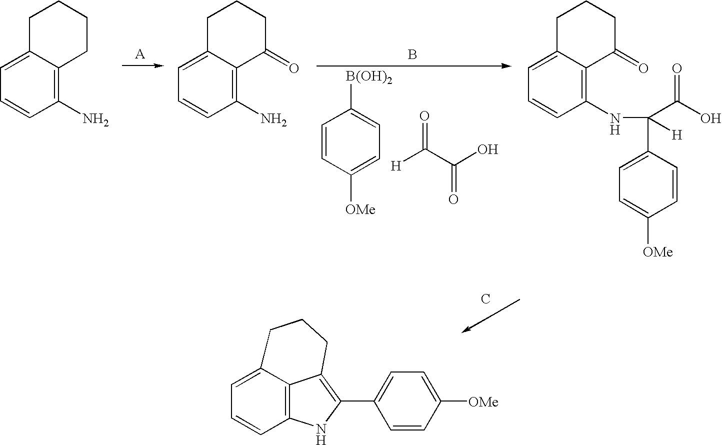

### Synthesis of 2-(4-methoxy-phenyl)-1,3,4,5-tetrahydro-benzo[cd]indole

Step A: To acetic anhydride (5 ml) in anhydrous ethanol (30 ml) at 0° C. 5,6,7,8-tetrahydro-naphthalen-1-ylamine (18 mmol, 2.5 ml) was added. The mixture was stirred at room temperature for 18 hours. The solvent was removed under reduced pressure to yield N-(5,6,7,8-tetrahydro-naphthalen-1-yl)-acetamide as white solid (3.4 g crude). The product was used without any further purification. To a solution of crude N-(5,6,7,8-tetrahydro-naphthalen-1-yl)-acetamide (3.4 g, 18 mmol) in acetone (50 ml) 15% aqueous MgSO₄ (3 g in 20 ml) was added followed by treatment with solid KMnO₄ at room temperature. The reaction mixture was allowed to stir at room temperature overnight. The brown mixture was filtered through Celite and the solids were washed with chloroform and water. The filtrate was extracted several times with chloroform. Organic layers were combined and washed with brine, dried and concentrated to give crude N-(8-oxo-5,6,7,8-tetrahydro-naphthalen-1-yl)-acetamide. The product was used for the next step without any purification. The crude N-(8-oxo-5,6,7,8-tetrahydro-naphthalen-1-yl)-acetamide was suspended in 6 N HCl and the reaction mixture was refluxed for 5 hours. After cooling to room temperature 2 N NaOH was added in small portions until the pH=8. The aqueous layer was extracted with ethyl acetate and organic layers were combined, washed with brine, dried, filtered, and concentrated. The residue was purified by flash chromatography (10% ethyl acetate:hexanes) to obtain of 8-amino-3,4-dihydro-2H-naphthalen-1-one in good yield (4 g, 48% over three steps). ¹H NMR (400 MHz, CDCl₃): δ7.16 (t, J=7.1 Hz, 1H), 6.50-6.43 (m, 2H), 6.45 (broad s, 2H, NH₂), 2.88 (t, J=6.7 Hz, 2H), 2.64 (t, J=5.7 Hz, 2H), 2.04 (m, 2H). ¹³C NMR (100 MHz, CDCl₃): δ201.3, 151.3, 146.0, 134.3, 115.8, 115.4, 114.7, 40.3, 30.9, 22.9.

Step B: To a solution of the product of Step A (1 eq., 1 mmol) and glyoxylic acid monohydrate (1 eq., 1 mmol, 92 mg) in acetonitrile, p-methoxyphenyl boronic acid (1 eq. 1 mmol) was added. The resulting reaction mixture was stirred at room temperature till TLC indicated that starting materials disappeared. The resulting mixture was concentrated under reduced pressure and the residue was purified via flash chromatography to afford (4-methoxy-phenyl)-(8-oxo-5,6,7,8-tetrahydro-naphthalen-1-ylamino)-acetic acid in good yield (76%). ¹H NMR (400 MHz, methanol-d₄): δ7.39 (d, J=8.5 Hz, 2H), 7.08 (m, 1H), 6.86 (d, J=8.6 Hz, 2H), 6.38 (d, J=7.7 Hz, 1H), 6.32 (d, J=8.6 Hz, 1H), 5.12 (s, 1H), 3.71 (s, 3H), 2.80 (m, 2H), 2.59 (m, 2H), 1.95 (m, 2H). ¹³C NMR (100 MHz, methanol-d₄): δ201.9, 173.0, 159.6, 149.5, 146.8, 135.2, 134.7, 129.8, 128.0, 115.3, 115.1, 113.8, 112.8, 110.2, 59.2, 54.3, 39.9, 30.6, 22.7.

Step C: To a suspension of the amino acid product of Step B (1 eq., 0.3 mmol) in toluene, p-toluenesulfonic acid chloride was added (1 eq., 0.3 mmol), followed by addition of triethylamine (2 eq., 0.6 mmol). The reaction mixture was stirred at room temperature for 4 hours and extracted with water and ethyl acetate. The organic residue was dried with sodium sulfate then purified on silica gel (ethyl acetate:hexane) to afford 2-(4-methoxy-phenyl)-1,3,4,5-tetrahydro-benzo[cd]indole in good yield (63%). ¹H NMR (400 MHz, CDCl₃): δ7.97 (broad s, 1H), 7.57 (d, J=8.7 Hz, 2H), 7.21-7.12 (m, 2H), 7.04 (d, J=9.1 Hz, 2H), 6.89 (d, J=6.8 Hz, 2H), 3.90 (s, 3H), 3.08 (t, J=6.1 Hz, 2H), 3.01 (t, J=6.1 Hz, 2H), 2.14 (m, 2H). ¹³C NMR (100 MHz, CDCl₃): δ158.7, 134.1, 132.1, 130.3, 128.9, 127.4, 126.3, 122.6, 116.2, 114.4, 110.3, 107.8, 55.4, 29.7, 27.5, 24.6, 23.1.

### Example 8

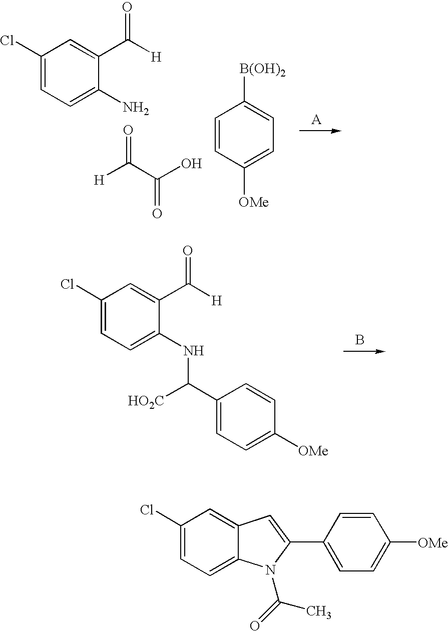

### Synthesis of 1-[5-chloro-2-(4-methoxy-phenyl)-indol-1-yl]-ethanone

Step A: To a solution of amine (1 eq., 1 mmol) and glyoxylic acid monohydrate (1 eq., 1 mmol, 92 mg) in acetonitrile, p-methoxyphenylboronic acid (1 eq., 1 mmol) was added. The resulting reaction mixture was stirred at room temperature till TLC indicated that starting materials disappeared. The resulting mixture was concentrated under reduced pressure and the residue was purified via flash chromatography to afford (4-chloro-2-formyl-phenylamino)-(4-methoxy-phenyl)-acetic acid in high yield (89%). ¹H NMR (400 MHz, methanol-d₄): δ9.79 (s, 1H), 7.52 (s, 1H), 7.38 (m, 2H), 7.18 (m, 1H), 6.88 (m, 2H), 6.48 (s, 1H), 5.16 (s, 1H), 3.74 (s, 3H). ¹³C NMR (400 MHz, methanol-d₄): δ193.3, 172.7, 159.7, 146.7, 135.1, 134.9, 129.3, 128.0, 119.9, 119.8, 113.9, 113.8, 58.9, 54.3.

Step B: In a 1 dram vial acetic anhydride as a solvent, triethylamine (0.5 ml) and (0.3 mmol) of amino acid product of Step B were mixed together. The reaction mixture was heated till 90° C. and let stirred at this temperature for 30 minutes. After the reaction was completed (no amino acid on TLC plate was left), the solvent was evaporated under reduced pressure. The residue was purified by flash chromatography (ethyl acetate:hexanes) to obtain 1-[5-chloro-2-(4-methoxy-phenyl)-indol-1-yl]-ethanone as a white solid in excellent yield (99%). ¹H NMR (400 MHz, CDCl₃): δ8.33 (d, J=8.9 Hz, 1H), 7.77 (s, 1H), 7.42-7.39 (m, 4H), 7.06 (d, J=9.3 Hz, 2H), 3.93 (s, 3H), 1.96 (s, 3H). ¹³C NMR (100 MHz, CDCl₃): δ171.5, 160.2, 140.9, 135.9, 130.4, 130.3, 129.0, 125.8, 124.8, 119.7, 117.2, 114.3, 110.0, 55.4, 27.8.

### Example 9

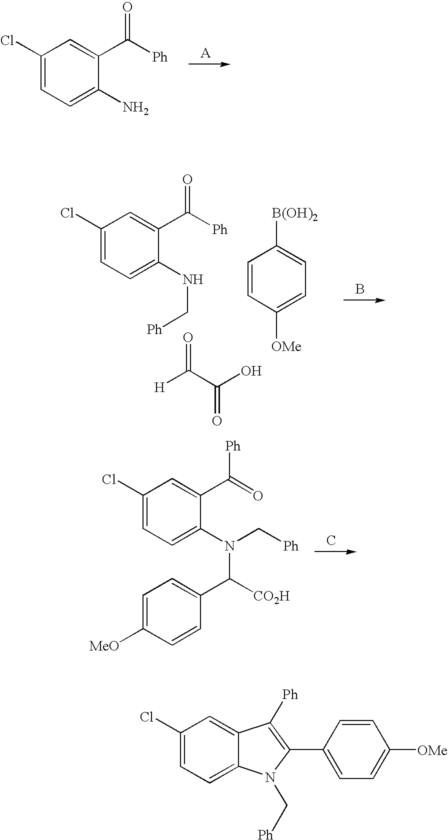

### Synthesis of 1-benzyl-5-chloro-2-(4-methoxy-phenyl)-3-phenyl-1H-indole

Step A: To a solution of (2-amino-5-chloro-phenyl)-phenyl-methanone (5 mmol, 1.16 g) in acetonitrile, cesium carbonate (5.5 mmol, 1.8 g) was added followed by addition of benzyl bromide (5.5 mmol, 0.65 ml). The reaction mixture was heated up to 60° C. and let stirred at this temperature for 12 hours. The solvent was evaporated and the residue was dissolved in water: ethyl acetate mixture. The organic layer was separated, concentrated under reduced pressure and the residue was purified via flash chromatography (ethyl acetate:hexanes) in order to obtain (2-benzylamino-5-chloro-phenyl)-phenyl-methanone as a yellow solid in good yield (750 mg, 47%). ¹H NMR (400 MHz, CDCl₃): δ9.00 (broad s, 1H), 7.70-7.28 (m, 12H), 6.72 (d, J=9.2 Hz, 1H), 4.52 (s, 2H). ¹³C NMR (100 MHz, CDCl₃): δ198.4, 150.0, 138.1, 134.8, 134.1, 131.3, 129.1, 128.8, 128.6, 128.4, 128.3, 127.4, 127.1, 118.9, 118.3.

Step B: To a solution of the product of Step A (1 eq., 1 mmol) and glyoxylic acid monohydrate (1 eq., 1 mmol, 92 mg) in acetonitrile, p-methoxyphenylboronic acid (1 eq., 1 mmol) was added. The resulting reaction mixture was stirred at room temperature till TLC indicated that starting materials disappeared. The resulting mixture was concentrated under reduced pressure and the residue was purified via flash chromatography to afford the crude amino acid.

Step C: In a 1 dram vial acetic anhydride as a solvent, triethylamine (0.5 ml) and amino acid product of Step B (0.3 mmol) were mixed together. The reaction mixture was heated up to 90° C. and let stirred at this temperature for 30 minutes. After the reaction was completed (no amino acid on TLC plate was left), the solvent was evaporated under reduced pressure. The residue was purified by flash chromatography (ethyl acetate:hexanes) to yield 1-benzyl-5-chloro-2-(4-methoxy-phenyl)-3-phenyl-1H-indole in good yield (51%).

¹H NMR (250 MHz, CDCl₃): δ7.77 (s, 1H), 7.32-7.14 (m, 12H), 7.00 (d, J=6.7 Hz, 2H), 6.84 (d, J=8.9 Hz, 2H), 5.27 (s, 2H), 3.80 (s, 3H). ¹³C NMR (100 MHz, CDCl₃): δ159.7, 139.1, 137.8, 135.3, 134.6, 132.2, 129.8, 128.8, 128.4, 127.4, 126.1, 126.0, 125.8, 123.4, 122.4, 119.0, 115.0, 114.0, 111.5, 55.1, 47.7.

### Example 10

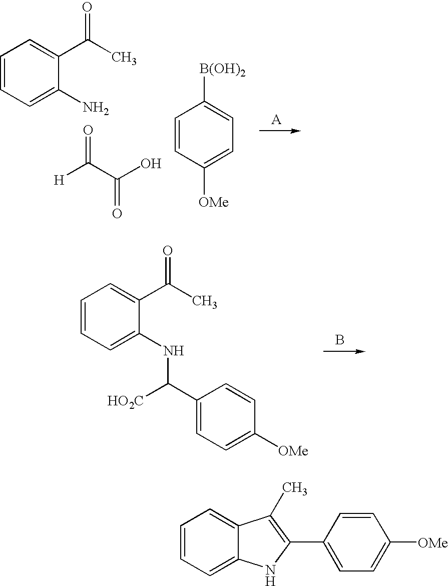

### Synthesis of 2-(4-methoxy-phenyl)-3-methyl-1H-indole

Step A: To a solution of commercially available amine (1 eq., 1 mmol) and glyoxylic acid monohydrate (1 eq., 1 mmol, 92 mg) in acetonitrile, p-methoxyphhenylboronic acid (1 eq., 1 mmol) was added. The resulting reaction mixture was stirred at room temperature till TLC indicated that starting materials disappeared. The resulting mixture was concentrated under reduced pressure and the residue was purified via flash chromatography to afford (2-acetyl-phenylamino)-(4-methoxy-phenyl)-acetic acid in excellent yield (98%). ¹H NMR (400 MHz, methanol-d₄): δ7.87 (m, 1H), 7.41 (d, J=8.3 Hz, 2H), 7.26 (t, J=8.3 Hz, 1H), 6.91 (d, J=8.4 Hz, 2H), 6.63 (t, J=7.7 Hz, 1H), 6.54 (d, J=8.2 Hz, 1H), 5.17 (s, 1H), 3.78 (s, 3H), 2.61 (s, 3H). ¹³C NMR (62.5 MHz, methanol-d₄): δ203.0, 174.6, 161.0, 150.1, 136.0, 134.0, 131.3, 129.4, 119.5, 116.2, 115.2, 114.0, 60.5, 55.7, 28.0.

Step B: To a suspension of the amino acid product of Step A (0.3 mmol) in toluene, p-toluenesulfonic acid chloride was added (1 eq., 0.3 mmol), followed by addition of triethylamine (2 eq., 0.6 mmol). The reaction mixture was stirred at room temperature for 4 hours and extracted with water and ethyl acetate. The organic residue was dried with sodium sulfate then purified on silica gel (ethyl acetate:hexane) to afford 2-(4-methoxy-phenyl)-3-methyl-1H-indole in good yield (46%). ¹H NMR (400 MHz, CDCl₃): δ7.97 (broad s, 1H), 7.58 (d, J=8.1 Hz, 1H), 7.50 (d, J=9.0 Hz, 2H), 7.34 (d, J=7.7 Hz, 1H), 7.20-7.12 (m, 2H), 7.00 (d, J=9.1 Hz, 2H), 3.86 (s, 3H), 2.43 (s, 3H). ¹³C NMR (100 MHz, CDCl₃): δ159.0, 129.0, 125.9, 121.9, 119.4, 118.7, 114.3, 110.5, 107.7, 55.4, 9.6.

### Example 11

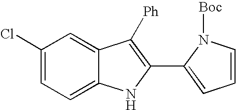

### Synthesis of 5′-Chloro-3′-phenyl-1′H-[2,2′]biindolyl-1-carboxylic acid tert-butyl ester

Prepared analogously to Example 10 in two step procedure from (2-amino-5-chloro-phenyl)-phenyl-methanone in good yield (81%). ¹H NMR (400 MHz, CDCl₃): δ8.38 (s, 1H), 8.12 (d, J=8.3 Hz, 1H), 7.69 (s, 1H), 7.39 (d, J=7.6 Hz, 2H), 7.32-7.11 (m, 8H). ¹³C NMR (100 MHz, CDCl₃): δ149.8, 137.0, 134.2, 130.1, 129.0, 128.8, 128.6, 128.4, 128.1, 126.4, 126.1, 125.1, 123.3, 123.2, 121.0, 119.4, 117.1, 115.5, 113.5, 111.9, 83.9, 27.7.

### Example 12

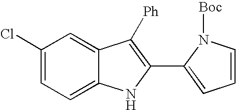

### Synthesis of 5′-chloro-3′-phenyl-1′H-[2,2′]biindolyl-1-carboxylic acid tert-butyl ester

Prepared analogously to Example 10 in two step procedure from (2-amino-5-chloro-phenyl)-phenyl-methanone in good yield (73%). ¹H NMR (400 MHz, CDCl₃): δ8.73 (broad s, 1H), 7.76 (s, 1H), 7.42-7.20 (m, 8H), 6.28 (m, 1H), 6.23 (t, J=3.0 Hz, 1H), 1.34 (s, 9H). ¹³C NMR (100 MHz, CDCl₃): δ149.0, 134.7, 133.9, 129.1, 128.5, 128.2, 128.0, 126.2, 125.8, 124.5, 123.1, 122.9, 119.1, 118.2, 117.1, 111.9, 111.1, 84.2, 27.6.

### Example 13

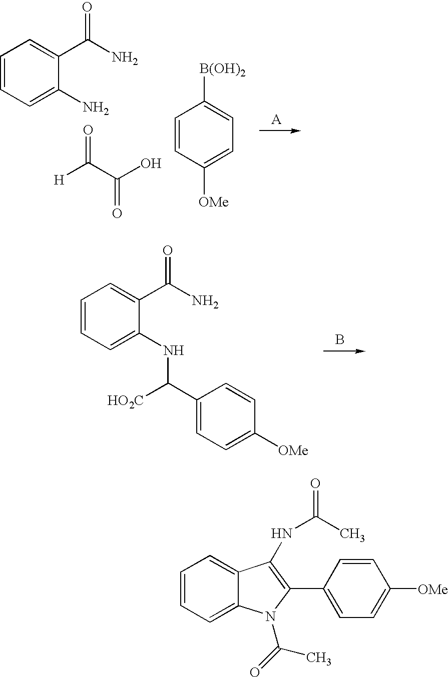

### Synthesis of N-[1-acetyl-2-(4-methoxy-phenyl)-1H-indol-3-yl]-acetamide

Step A: To a solution of 2-amino-benzamide (1 eq., 1 mmol,) and glyoxylic acid monohydrate (1 eq., 1 mmol, 92 mg) in acetonitrile, p-methoxyphenylboronic acid (1 mmol) was added. The resulting reaction mixture was stirred at room temperature till TLC indicated that starting materials disappeared. The resulting mixture was concentrated under reduced pressure and the residue was purified via flash chromatography to afford (2-carbamoyl-phenylamino)-(4-methoxy-phenyl)-acetic acid in good yield (84%). ¹H NMR (400 MHz, methanol-d₄): δ7.11 (d, J=6.3 Hz, 1H), 7.44 (d, J=5.4 Hz, 2H), 7.21 (t, J=6.6 Hz, 1H), 6.93 (d, J=8.5 Hz, 2H), 6.64 (t, J=7.9 Hz, 1H), 6.54 (d, J=8.5 Hz, 1H), 5.13 (s, 1H), 3.80 (ds, 3H). ¹³C NMR (62.5 MHz, methanol-d₄): δ174.8, 161.2, 148.9, 133.9, 131.3, 130.1, 129.6, 116.9, 115.2, 114.1, 60.9, 55.8.

Step B: In a 1 dram vial acetic anhydride as a solvent, triethylamine (0.5 ml) and amino acid product of Step B (0.3 mmol) were mixed together. The reaction mixture was heated till 90° C. and let stirred at this temperature for 30 minutes. After the reaction was completed (no amino acid on TLC plate was left), the solvent was evaporated under reduced pressure. The residue was purified by flash chromatography (ethyl acetate:hexanes) to yield N-[1-acetyl-2-(4-methoxy-phenyl)-1H-indol-3-yl]-acetamide in good yield (79%). ¹H NMR (400 MHz, CDCl₃): δ8.45 (d, J=8.3 Hz, 1H), 7.41-7.29 (m, 5H), 7.01 (d, J=8.4 Hz, 2H), 3.88 (s, 3H), 2.25 (s, 3H), 2.03 (s, 3H). ¹³C NMR (100 MHz, CDCl₃): δ171.2, 169.3, 160.4, 134.9, 133.3, 131.1, 128.2, 126.0, 123.8, 123.1, 122.6, 117.2, 116.6, 114.4, 55.2, 27.7, 20.

### Example 14

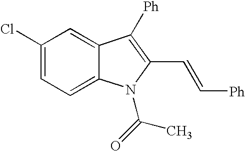

### Synthesis of 1-(5-chloro-3-phenyl-2-styryl-indol-1-yl)-ethanone

Prepared analogously to Example 10 using styryl boronic acid in two step procedure in high yield (90%). ¹H NMR (400 MHz, CDCl₃): δ8.24 (d, J=9.0 Hz, 1H), 7.52-7.29 (m, 12H), 7.22 (d, J=16.4 Hz, 1H), 6.62 (d, J=16.4 Hz, 1H), 2.72 (s, 3H). ¹³C NMR (100 MHz, CDCl₃): δ171.2, 136.4, 136.3, 135.0, 134.8, 132.9, 131.2, 130.1, 129.4, 128.9, 128.8, 128.5, 127.6, 126.5, 125.5, 123.0, 119.2, 118.3, 116.7, 28.1.

### Example 15

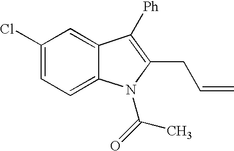

### Synthesis of 1-(2-allyl-5-chloro-3-phenyl-indol-1-yl)-ethanone

Prepared analogously to Example 10 using allyl pinacol boronate in two step procedure in good yield (46%). ¹H NMR (400 MHz, CDCl₃): δ7.86 (d, J=8.9 Hz, 1H), 7.42-7.32 (m, 6H), 7.19 (dd, J=8.9 Hz, J=2.9 Hz, 1H), 6.00-5.91 (m, 1H), 5.05 (d, J=12.4 Hz, 1H), 4.84 (d, J=19.5, 1H), 3.70 (m, 2H), 2.70 (s, 3H). ¹³C NMR (100 MHz, CDCl₃): δ170.1, 136.0, 135.7, 134.3, 132.6, 131.5, 129.8, 129.0, 128.8, 127.8, 124.4, 123.1, 119.2, 116.4, 116.3, 31.3, 27.2.

### Example 16

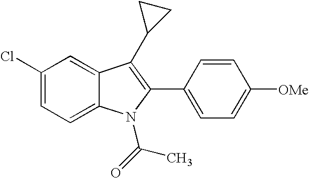

### Synthesis of 1-[3-cyclopropyl-2-(4-methoxy-phenyl)-indol-1-yl]-ethanone

Prepared analogously to Example 10 in a two step procedure in good yield as a major product (42%). ¹H NMR (400 MHz, CDCl₃): δ8.44 (d, J=7.4 Hz, 1H), 7.67 (d, J=8.3 Hz, 1H), 7.39 (d, J=8.8 Hz, 3H), 7.36-7.30 (m, 2H), 7.04 (d, J=7.7 Hz, 2H), 3.93 (s, 3H), 2.00 (s, 3H), 1.82-1.72 (m, 1H), 0.80-0.75 (m, 2H), 0.60-0.56 (m, 2H). ¹³C NMR (100 MHz, CDCl₃): δ171.5, 159.7, 136.7, 136.1, 131.7, 129.7, 125.8, 124.9, 123.3, 122.0, 119.1, 116.3, 114.0, 55.3, 27.8, 6.6, 5.7.

### Example 17

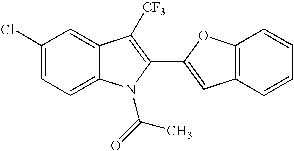

### Synthesis of 1-(2-benzofuran-2-yl-5-chloro-3-trifluoromethyl-indol-1-yl)-ethanone

Prepared analogously to Example 10 in a two step procedure in moderate yield as a minor product (23%) along with major product 2-benzofuran-2-yl-5-chloro-3-trifluoromethyl-1H-indole in good yield (52%). ¹H NMR (400 MHz, CDCl₃) minor product: δ8.36 (d, J=9.6 Hz, 1H), 7.81 (s, 1H), 7.72 (d, J=7.7 Hz, 1H), 7.59 (d, J=8.2 Hz, 1H), 7.47-7.44 (m, 2H), 7.39-7.35 (m, 1H), 7.22 (s, 1H), 2.12 (s, 3H). ¹⁹F NMR (376 MHz, CDCl₃): δ-55.1. ¹³C NMR (100 MHz, CDCl₃): δ170.4, 155.3, 142.7, 134.7, 130.5, 128.8, 127.6, 127.5, 126.4, 125.4, 124.0, 122.1, 121.5, 119.8, 117.5, 114.0 (q, J=35.9 Hz), 111.8, 25.0. ¹H NMR (400 MHz, CDCl₃) major product: δ9.13 (s, 1H), 7.73 (s, 1H), 7.58 (d, J=7.2 Hz, 1H), 7.44 (d, J=7.9 Hz, 1H), 7.32-7.17 (m, 5H). ¹⁹F NMR (376 MHz, CDCl₃): δ-54.9. ¹³C NMR (100 MHz, CDCl₃): δ154.3, 144.9, 132.9, 128.9, 127.8, 126.5, 125.7, 124.6, 123.7, 122.0, 121.5, 119.6, 112.4, 111.5, 111.1, 107.8.

### Example 18

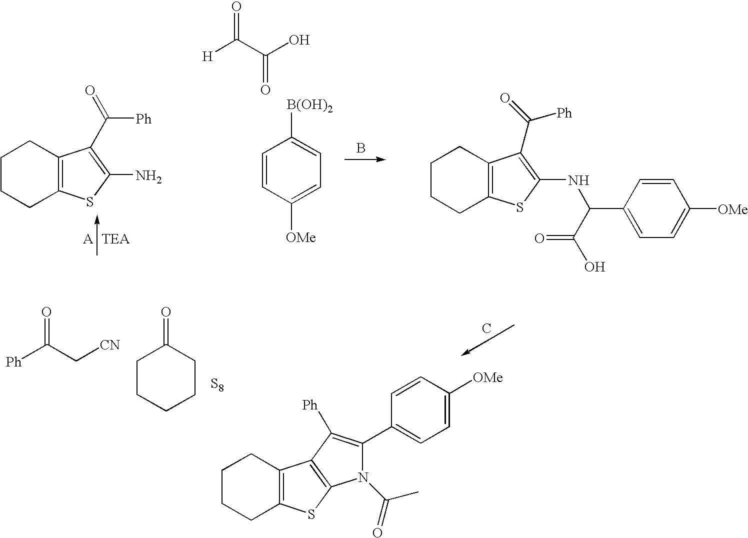

### Synthesis of 1-(3-phenyl-2-styryl-4,5,6,7-tetrahydro-8-thia-1-aza-cyclopenta[α]inden-1-yl)-ethanone

Step A: This is prepared using the Gewald reaction. To a solution of 3-oxo-3-phenyl-propionitrile (5 mmol, 725 mg), cyclohexanone (5 mmol, 0.52 ml) and triethylamine (5 mmol, 0.44 ml) in 10 ml of ethanol, pulverized sulfur (5 mmol, 164 mg) was added. The reaction mixture was refluxed for two hours. The solvent was evaporated and the residue was washed with water and extracted with ethyl acetate. The organic layer was dried with sodium sulfate and the volatiles removed under reduced pressure. The residue was purified on silica (25% ethyl acetate:hexanes) in order to isolate 1.08 g of (2-amino-4,5,6,7-tetrahydro-benzo[b]thiophen-3-yl)-phenyl-methanone as a yellow solid (84% yield). ¹H NMR (400 MHz, CDCl₃): δ7.48-7.37 (m, 5H), 6.47 (broad s, 2H), 2.50 (m, 2H), 1.79 (m, 2H), 1.72 (m, 2H), 1.49-1.43 (m, 2H). ¹³C NMR (100 MHz, CDCl₃): δ192.7, 164.5, 142.2, 131.3, 130.3, 128.0, 127.5, 118.5, 115.9, 27.8, 24.7, 23.1, 22.9.

Step B: To a solution of (2-amino-4,5,6,7-tetrahydro-benzo[b]thiophen-3-yl)-phenyl-methanone (1 mmol, 257 mg) the product of Step A and glyoxylic acid monohydrate (1 mmol, 92 mg) in 2 ml of acetonitrile, 1 mmol of styryl boronic acid (147 mg) was added. The resulting reaction mixture was stirred at room temperature till TLC indicated that starting materials disappeared. The resulting suspension was concentrated under reduced pressure and the residue was purified via flash chromatography (ethyl acetate: methanol: ammonia) to afford 2-(3-benzoyl-4,5,6,7-tetrahydro-benzo[b]thiophen-2-ylamino)-4-phenyl-but-3-enoic acid as a yellow solid in good yield (346 mg, 83%).

Step C: In a 1 dram vial acetic anhydride as a solvent, triethylamine (0.5 ml) and (0.3 mmol, 126.3 mg) of amino acid product of Step B were mixed together. The reaction mixture was heated till 90° C. and let stirred at this temperature for 30 minutes. After the reaction was completed (no amino acid on TLC plate was left), the solvent was evaporated under reduced pressure. The residue is purified by flash chromatography 10% ethyl acetate:hexanes to yield 1-(3-phenyl-2-styryl-4,5,6,7-tetrahydro-8-thia-1-aza-cyclopenta[α]inden-1-yl)-ethanone as a yellow solid (76%). ¹H NMR (400 MHz, CDCl₃): δ7.46-7.22 (m, 11H), 6.36 (d, J=16.1 Hz, 1H), 2.85-2.82 (m, 2H), 2.72 (s, 3H), 2.33-2.30 (m, 2H), 1.91-1.85 (m, 2H), 1.75-1.70 (m, 2H). ¹³C NMR (100 MHz, CDCl₃): δ168.5, 137.4, 134.7, 133.4, 133.0, 132.4, 131.7, 131.2, 130.3, 128.6, 128.2, 127.7, 127.2, 126.2, 125.6, 124.1, 118.8, 25.5, 25.4, 25.1, 23.5, 22.6.

### Example 19

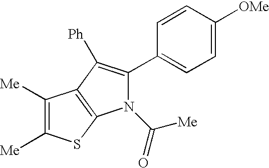

### Synthesis of (2-amino-4,5-dimethyl-thiophen-3-yl)-phenyl-methanone

Prepared analogously to Example 18. ¹H NMR (400 MHz, CDCl₃): δ7.30-7.23 (m, 7H), 6.87 (d, J=9.5 Hz, 2H), 3.83 (s, 3H), 2.44 (s, 3H), 2.02 (s, 3H), 1.97 (s, 3H). ¹³C NMR (100 MHz, CDCl₃): δ168.5, 159.7, 133.8, 132.9, 130.9, 130.8, 130.5, 130.2, 129.7, 127.7, 126.7, 124.8, 124.4, 122.7, 122.3, 113.8, 55.2, 25.0, 13.3, 12.6.

### Example 20

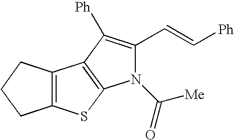

### Synthesis of 1-(3-Phenyl-2-styryl-5,6-dihydro-4H-7-thia-1-aza-cyclopenta[α]pentalen-1-yl)-ethanone

Prepared analogously to Example 18. ¹H NMR (400 MHz, CDCl₃): δ7.40-7.38 (m, 2H), 7.29 (t, J=7.3 Hz, 2H), 7.22-7.14 (m, 6H), 6.38 (d, J=16.0 Hz, 1H), 2.86 (t, J=7.1 Hz, 2H), 2.60 (s, 3H), 2.55 (t, J=6.7 Hz, 2H), 2.35-2.27 (m, 2H). ¹³C NMR (100 MHz, CDCl₃): δ168.5, 139.1, 137.1, 136.9, 134.7, 134.2, 131.8, 131.7, 129.8, 128.7, 128.3, 127.9, 127.0, 126.8, 126.3, 123.4, 118.8, 29.2, 28.5, 25.7.

### Example 21

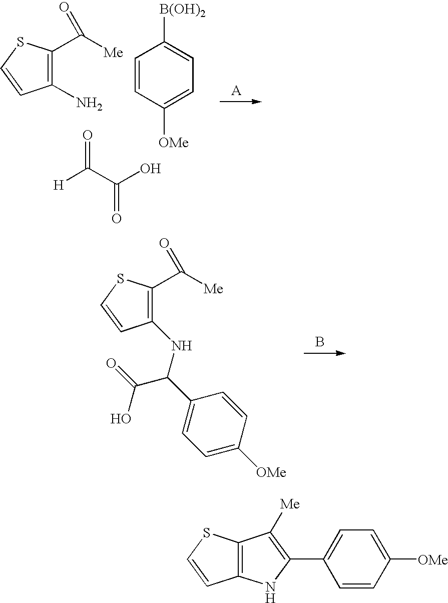

### Synthesis of 1-[5-(3,4-dimethyl-phenyl)-6-methyl-thieno[3,2-b]pyrrol-4-yl]-ethanone

Step A: To a solution of 1-(3-amino-thiophen-2-yl)-ethanone (1 mmol) and glyoxylic acid monohydrate (1 mmol, 92 mg) in 2 ml of acetonitrile, 1 mmol of p-methoxy phenylboronic acid (1 mmol, 152 mg) was added. The resulting reaction mixture was stirred at room temperature till TLC indicated that starting materials disappeared. The resulting suspension was filtered off and the solid was evaporated few times with methanol in order to get rid of boric acid. (2-Acetyl-thiophen-3-ylamino)-(4-methoxy-phenyl)-acetic acid was isolated as a yellow solid in good yield (82%). ¹H NMR (400 MHz, acetone-d₆): δ7.54 (d, J=6.0 Hz, 1H), 7.47 (d, J=8.4 Hz, 2H), 6.98 (d, J=9.4 Hz, 2H), 6.67 (d, J=5.3 Hz, 1H), 5.38 (s, 1H), 3.82 (s, 3H), 2.36 (s, 3H). ¹³C NMR (100 MHz, acetone-d₆): δ190.7, 172.4, 160.9, 154.5, 133.3, 131.2, 129.3, 118.4, 115.1, 112.1, 61.2, 55.7, 28.5.

Step B: In a 1 dram vial acetic anhydride as a solvent, triethylamine (0.5 ml) and (0.3 mmol) of amino acid (the product of Step A) were mixed together. The reaction mixture was heated till 90° C. and let stirred at this temperature for 30 minutes. After the reaction was completed (no amino acid on TLC plate was left), the solvent was evaporated under reduced pressure. The residue was purified by flash chromatography (ethyl acetate:hexanes) to obtain the product in moderate yield (40%) as a major product along with 5-(3,4-dimethyl-phenyl)-6-methyl-4H-thieno[3,2-b]pyrrole (24%). ¹H NMR (400 MHz, CDCl₃) major product: δ7.66 (d, J=5.1 Hz, 1H), 7.31 (d, J=8.6 Hz, 2H), 7.24 (d, J=5.1 Hz, 1H), 7.03 (d, J=8.2 Hz, 2H), 3.91 (s, 3H), 2.09 (s, 3H), 2.04 (s, 3H). ¹³C NMR (100 MHz, CDCl₃): δ168.9, 159.7, 138.5, 133.6, 131.9, 128.7, 125.6, 124.2, 116.9, 116.8, 114.1, 55.3, 26.0, 11.0. ¹H NMR (400 MHz, CDCl₃) minor product: δ7.98 (broad s, 1H), 7.33 (d, J=8.5 Hz, 2H), 7.18 (s, 1H), 6.98 (d, J=5.2 Hz, 1H), 6.91 (d, J=8.5 Hz, 2H), 6.87 (d, J=5.0 Hz, 1H), 3.78 (s, 3H), 2.29 (s, 3H). ¹³C NMR (100 MHz, CDCl₃): δ158.5, 137.0, 133.3, 128.4, 127.0, 126.4, 122.8, 114.3, 111.3, 108.2, 55.3, 29.7, 11.5.

### Example 22

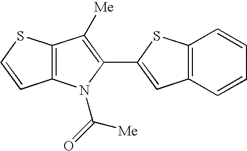

### Synthesis of 1-(5-benzo[b]thiophen-2-yl-6-methyl-thieno[3,2-b]pyrrol-4-yl)-ethanone

Prepared analogously to Example 21. ¹H NMR (400 MHz, CDCl₃): δ7.91-7.88 (m, 2H), 7.67 (d, J=5.1 Hz, 1H), 7.48-7.42 (m, 2H), 7.37 (s, 1H), 7.33 (d, J=5.0 Hz, 1H), 2.30 (s, 3H), 2.21 (s, 3H). ¹³C NMR (100 MHz, CDCl₃): δ168.6, 141.2, 139.6, 139.3, 134.4, 128.7, 127.0, 125.8, 125.4, 125.1, 124.8, 124.1, 122.3, 120.5, 116.9, 25.1, 11.3.

### Example 23

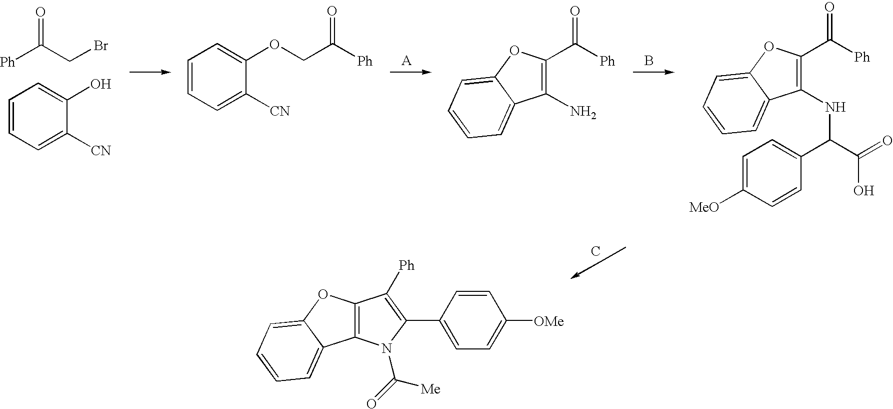

### Synthesis of 1-[2-(4-Methoxy-phenyl)-1-phenyl-8-oxa-3-aza-cyclopenta[α]inden-3-yl]-ethanone

Step A: To a solution of 2-hydroxy-benzonitrile (10 mmol, 1.19 g) in acetone (5 ml), 2-bromo-1-phenyl-ethanone (11 mmol, 2.2 g) and anhydrous potassium carbonate (2.2 eq., 22 mmol, 3.04 g) were added. The reaction was refluxed for 8 hours. The solid was filtered off and washed with lots of acetone 200 ml. The filtrate was concentrated under the reduced pressure to obtain (3-amino-benzofuran-2-yl)-phenyl-methanone as a yellow solid in good yield (66%, 1.55 g). ¹H NMR (400 MHz, CDCl₃): δ8.17-8.15 (m, 2H), 7.56 (d, J=7.4 Hz, 1H), 7.49-7.37 (m, 5H), 7.21-7.17 (m, 1H), 5.99 (broad s, 2H). ¹³C NMR (100 MHz, CDCl₃): δ183.0, 154.4, 142.4, 137.6, 135.0, 131.7, 129.8, 129.1, 128.2, 122.2, 120.7, 120.3, 112.5.

Step B: To a solution of the product from Step A (1 mmol) and glyoxylic acid monohydrate (1 mmol, 92 mg) in 2 ml of acetonitrile, p-methoxyphenylboronic acid (1 mmol) was added. The resulting reaction mixture was stirred at room temperature till TLC indicated that starting materials disappeared. The resulting suspension was filtered off and the solid was evaporated few times with methanol in order to get rid of boric acid. (2-Benzoyl-benzofuran-3-ylamino)-(4-methoxy-phenyl)-acetic acid was isolated in good yield as a brown solid (55%). ¹H NMR (400 MHz, acetone-d₆): δ8.30 (d, J=6.1 Hz, 2H), 7.98 (d, J=8.5 Hz, 1H), 7.68-7.53 (m, 7H), 7.27-7.22 (m, 1H), 6.99 (d, J=8.3 Hz, 2H), 6.04 (s, 1H), 3.80 (s, 3H). ¹³C NMR (62.5 MHz, acetone-d₆): δ182.5, 172.4, 160.8, 155.7, 143.4, 139.1, 132.6, 130.8, 130.0, 129.2, 124.7, 123.4, 120.9, 115.2, 113.6, 60.7, 55.6.

Step C: In a 1 dram vial acetic anhydride as a solvent, triethylamine (0.5 ml) and (0.3 mmol) of amino acid (product of Step B) were mixed together. The reaction mixture was heated till 90° C. and let stirred at this temperature for 30 minutes. After the reaction was completed (no amino acid on TLC plate was left), the solvent was evaporated under reduced pressure. The residue was purified by flash chromatography (ethyl acetate:hexanes) to obtain 1-[2-(4-methoxy-phenyl)-1-phenyl-8-oxa-3-aza-cyclopenta [α]inden-3-yl]-ethanone in good yield as a yellow solid (75%). ¹H NMR (400 MHz, CDCl₃): δ8.30 (d, J=9.3 Hz, 1H), 7.62 (d, J=9.0 Hz, 1H), 7.47-7.23 (m, 9H), 7.04 (d, J=7.7 Hz, 2H), 3.92 (s, 3H), 2.06 (s, 3H). ¹³C NMR (100 MHz, CDCl₃): δ169.6, 160.4, 159.0, 149.3, 132.9, 131.7, 131.5, 128.4, 128.3, 127.0, 125.1, 123.7, 123.0, 122.3, 121.4, 120.2, 114.6, 114.1, 112.0, 55.4, 26.2.

### Example 24

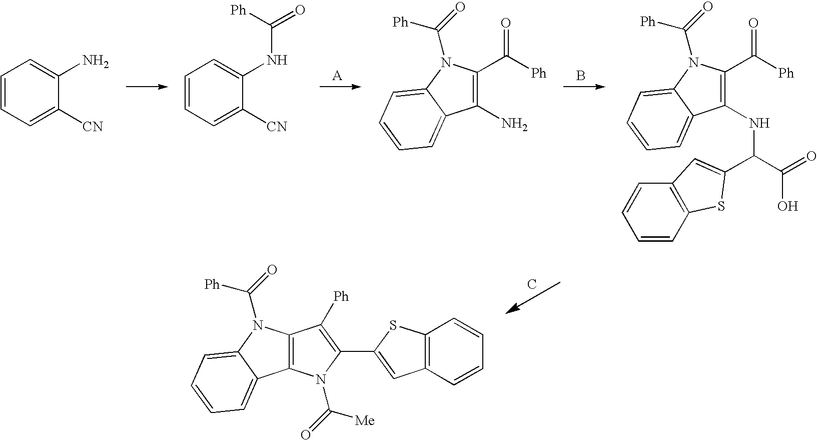

### Synthesis of 1-(2-benzo[b]thiophen-2-yl-8-benzoyl-1-phenyl-8H-3,8-diaza-cyclopenta[α]inden-3-yl)-ethanone

Step A: To a solution of 2-amino-benzonitrile (10 mmol, 1.18 g) and triethylamine (15 mmol, 2.1 ml), benzoyl chloride (11 mmol, 1.28 ml) was added at room temperature. The reaction was stirred at room temperature for 3 days. The solvent was evaporated, and the residue was purified via flash chromatography (10% ethyl acetate: 90% hexanes) to obtain N-(2-cyano-phenyl)-benzamide as a white solid in excellent yield (91%, 2 g). ¹H NMR (400 MHz, CDCl₃): δ8.60 (d, J=8.0 Hz, 1H), 8.42 (broad s, 1H), 7.94 (d, J=7.9 Hz, 1H), 7.68-7.51 (m, 5H), 7.22 (t, J=7.7 Hz, 1H). ¹³C NMR (100 MHz, CDCl₃): δ165.5, 140.7, 134.4, 133.7, 132.7, 132.2, 129.1, 127.2, 124.3, 121.2, 116.5, 102.2.

Step B: To a solution of N-(2-cyano-phenyl)-benzamide (10 mmol) in acetone (5 ml), 2-bromo-1-phenyl-ethanone (11 mmol, 2.2 g) and anhydrous potassium carbonate (2.2 eq., 22 mmol, 3.04 g) were added. The reaction was refluxed for 8 hours. The solid was filtered off and washed with lots of acetone 200 ml. The filtrate was concentrated under the reduced pressure to obtain (3-amino-1-benzoyl-1H-indol-2-yl)-phenyl-methanone as a yellow solid in good yield (71%). ¹H NMR (400 MHz, CDCl₃): δ8.18 (d, J=8.0 Hz, 1H), 7.62 (d, J=8.2 Hz, 1H), 7.50 (t, J=8.1 Hz, 1H), 7.31-7.23 (m, 3H), 7.12-7.02 (m, 8H), 5.86 (broad s, 2H). ¹³C NMR (100 MHz, CDCl₃): δ186.9, 173.0, 142.8, 138.5, 134.4, 134.0, 133.9, 133.7, 133.4, 132.8, 131.5, 130.4, 129.9, 129.2, 128.9, 128.5, 128.1, 123.6, 121.5, 119.5, 116.0, 112.5.

Step C: It is prepared in a two-step procedure. The first step was the amino acid synthesis of benzo[b]thiophen-2-yl-(1,2-dibenzoyl-1H-indol-3-ylamino)-acetic acid from the product of Step B in good yield (63%). 1-(2-Benzo[b]thiophen-2-yl-8-benzoyl-1-phenyl-8H-3,8-diaza-cyclopenta [α]inden-3-yl)-ethanone was isolated in good yield (51%).

### Example 25

### Activity of 1-[5-chloro-2-(4-methoxy-phenyl)-3-trifluoromethyl-indol-1-yl]-ethanone as a cytotoxic agent against several cancer cell lines

IC₅₀ was determined using an MTT assay. Modeling of this compound showed strong affinity to the active site of integrin alpha v beta 3 (αvβ₃), the vitronectin receptor that is expressed in activated endothelial cells, melanoma, and glioblastomas.

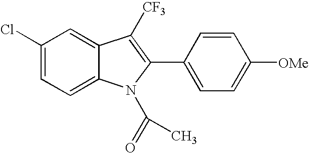

| HCT116 p53 ^{+/+} | HCT116 p53 ^{-/-} | MDA-MB 435 | H29 | Skov-3 | MCF7 |
|---|---|---|---|---|---|
| 14.5; 15.4 | 13.2; 8.4; 9.4; | 11.1; 8.5; 7.4, 9.6 | 9.6; 9.8; | 15.8; 17.2 | 18.8; 18 |
| (μg/mL) | 9.2 | (μg/mL) | (μg/mL) | (μg/mL) | (μg/mL) |
| | (μg/mL) | | | | |

### REFERENCES

Sundberg, R. J. (1996) Indoles, Academic Press Ltd, San Diego.
Humphrey, G. R.; Kuethe, J. T. Practical Methodologies for the Synthesis of Indoles. Chem. Rev. 2006, 106, 2875-2911.
Petasis, N. A.; Goodman, A.; Zavialov, I. A. A new synthesis of α-arylglycines from aryl boronic acids. Tetrahedron 1997, 53, 16463-16470.
Petasis, N. A.; Zavialov, I. A. A New and practical synthesis of α-amino acids from alkenyl boronic acids. J. Am. Chem. Soc. 1997, 119, 445-446.
Petasis, N. A.; Zavialov, I. A. Method for the synthesis of amines and amino acids with organoboron derivatives. U.S. Pat. No. 6,232,467, May 15, 2001.

What is claimed is:

1. A nitrogen heterocycle compound of formula 1: 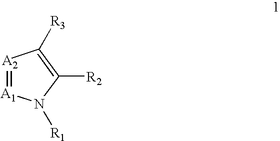
wherein:
R₁ is selected from the group consisting of H, alkyl, allyl, aryl, heteroaryl, acyl, trifluoroacyl, arylacyl, heteroarylacyl, pent-4-enylacyl, alkoxyacyl, allyloxyacyl, aryloxyacyl, aminoacyl, alkylaminoacyl, dialkylaminoacyl, arylmethyl, triarylmethyl, alkylsulfinyl, arylsulfinyl, alkylsulfonyl, arylsulfonyl, trialkylsilyl, aryldialkylsilyl, diarylalkylsilyl, bis(trimethylsilyl)methyl, and trialkylsilyl-ethanesulfonyl;
R₂ is selected from the group consisting of H, alkyl, allyl, alkenyl, alkynyl, allenyl, aryl, heteroaryl, trifluoromethyl, difluoromethyl, fluooroalkyl, difluoroalkyl, trifluoroalkyl, polyfluoroalkyl, acyl, carboxyl, alkoxyacyl, aryloxyacyl, aminoacyl, alkylaminoacyl, dialkylaminoacyl, and arylmethyl;
R₃ is selected from the group consisting of H, alkyl, allyl, aryl, heteroaryl, trifluoromethyl, 2,2,2-trifluoroethyl, fluooroalkyl, difluoroalkyl, trifluoroalkyl, polyfluoroalkyl, acyl, trifluoroacyl, arylacyl, carboxyl, alkoxyacyl, aryloxyacyl, aminoacyl, alkylaminoacyl, dialkylaminoacyl, amino, acylamino, alkoxyacylamino, aminoacylamino, alkylamino, dialkylamino, arylamino, arylalkylamino, and diarylamino;
A₁ and A₂ are independently selected from the group consisting of N and C—R, wherein R is selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, aminoacyl, alkylaminoacyl, dialkylaminoacyl, fluoro, bromo, iodo, hydroxy, alkoxy, aryloxy, cyano, amino, alkylamino, dialkylamino, arylamino, arylalkylamino, and diarylamino, and wherein groups A₁ and A₂ can be joined together to form a carbocyclic, heterocyclic, aromatic, or heteroaromatic ring; and
any two of R₁-R₃ and A₁-A₂ can be joined together to form a carbocyclic, heterocyclic, aromatic, or heteroaromatic ring.

2. A compound of claim 1, selected from the group consisting of compounds 2-29: 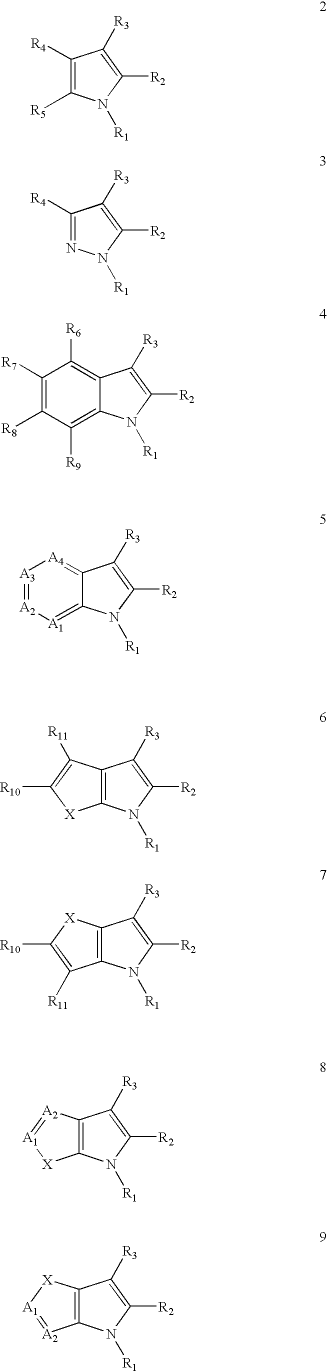 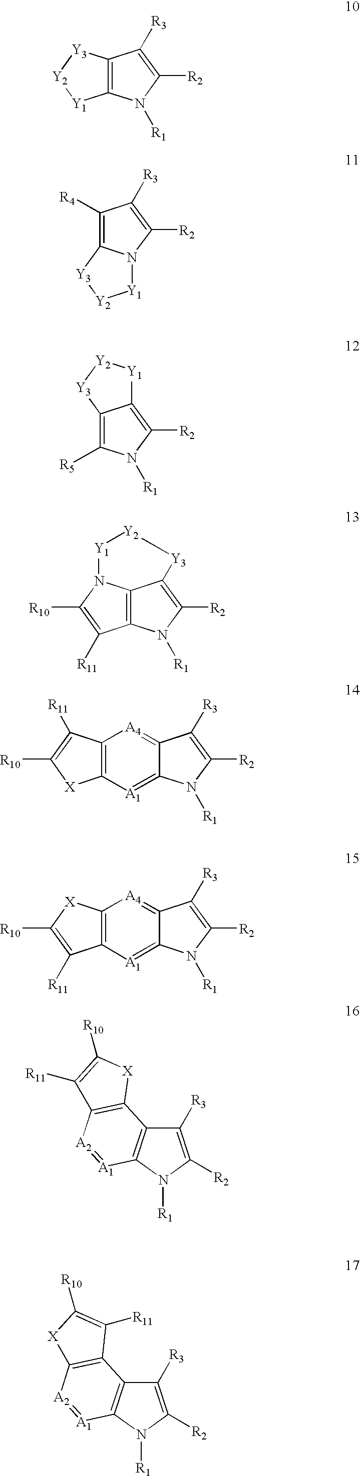 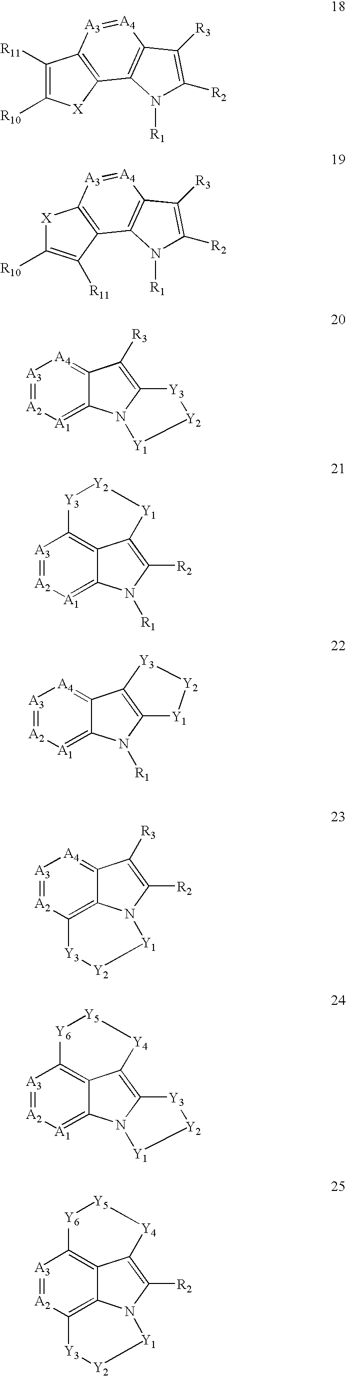 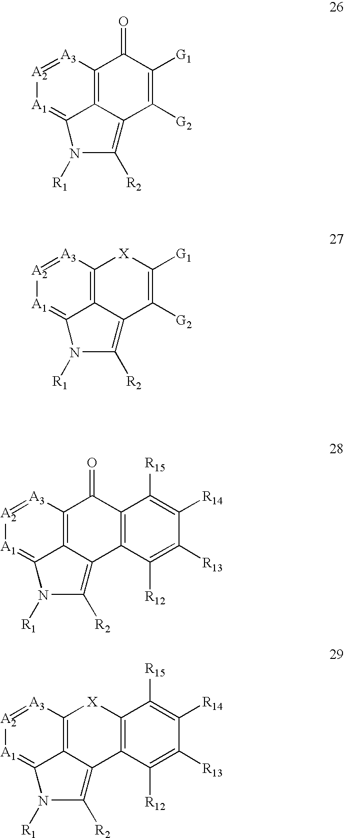
wherein:
R₁-R₃ and A₁-A₂ are defined as in claim 1;
R₄, R₆-R₉, and R₁₁-R₁₅ are independently selected from the group consisting of H, alkyl, allyl, aryl, heteroaryl, trifluoromethyl, 2,2,2-trifluoroethyl, fluooroalkyl, difluoroalkyl, trifluoroalkyl, polyfluoroalkyl, acyl, trifluoroacyl, arylacyl, carboxyl, alkoxyacyl, aryloxyacyl, fluoro, chloro, bromo, iodo, hydroxy, alkoxy, aryloxy, cyano, amino, acylamino, alkoxyacylamino, aminoacylamino, alkylamino, dialkylamino, arylamino, arylalkylamino, and diarylamino;
R₅ and R₁₀ are independently selected from the group consisting of H, alkyl, allyl, alkenyl, alkynyl, allenyl, aryl, heteroaryl, trifluoromethyl, fluooroalkyl, difluoroalkyl, trifluoroalkyl, polyfluoroalkyl, acyl, trifluoroacyl, arylacyl, carboxyl, alkoxyacyl, aryloxyacyl, fluoro, chloro, bromo, iodo, acyl, carboxyl, alkoxyacyl, aryloxyacyl, aminoacyl, alkylaminoacyl, arylaminoacyl, and dialkylaminoacyl;
A₃-A₄ are independently selected from the group consisting of N and C—R, wherein R is selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, fluoro, chloro, bromo, iodo, hydroxy, alkoxy, aryloxy, cyano, amino, alkylamino, dialkylamino, arylamino, arylalkylamino, diarylamino, aminoacyl, alkylaminoacyl, arylaminoacyl, and dialkylaminoacyl, and wherein there are no more than two Ns among A₁, A₂, A₃, and A₄, and wherein any two of A₁-A₄ can be joined together to form a carbocyclic, heterocyclic, aromatic, or heteroaromatic ring;
X is selected from the group consisting of O, S, and NRa, wherein Ra is selected from the group consisting of H, alkyl, allyl, aryl, heteroaryl, acyl, trifluoroacyl, arylacyl, heteroarylacyl, pent-4-enylacyl, alkoxyacyl, allyloxyacyl, aryloxyacyl, aminoacyl, alkylaminoacyl, dialkylaminoacyl, arylmethyl, triarylmethyl, alkylsulfinyl, arylsulfinyl, alkylsulfonyl, arylsulfonyl, alkylsulfinyl, arylsulfinyl, alkylsulfonyl, arylsulfonyl, trialkylsilyl, aryldialkylsilyl, diarylalkylsilyl, bis(trimethylsilyl)-methyl, and trialkylsilylethanesulfonyl;
Y₁-Y₂-Y₃ and Y₄-Y₅-Y₆ are independently a chain of 3-20 atoms selected from the group consisting of carbon, nitrogen, oxygen, and sulfur atoms;
G₁ and G₂ are independently selected from the group consisting of H, alkyl, allyl, aryl, heteroaryl, trifluoromethyl, fluooroalkyl, difluoroalkyl, trifluoroalkyl, polyfluoroalkyl, acyl, trifluoroacyl, arylacyl, alkoxyacyl, aryloxyacyl, aminoacyl, alkylaminoacyl, dialkylaminoacyl, fluoro, bromo, iodo, hydroxy, alkoxy, aryloxy, cyano, amino, alkylamino, dialkylamino, arylamino, arylalkylamino, and diarylamino, and wherein G₁ and G₂ can be joined together to form a carbocyclic, heterocyclic, aromatic, or heteroaromatic ring; and
any two of R₁-R₁₅, A₁-A₄, and G₁-G₂ can be joined together to form a carbocyclic, heterocyclic, aromatic, or heteroaromatic ring.

3. A compound of claim 2, wherein the Y₁-Y₂-Y₃ or Y₄-Y₅-Y₆ chain contains one or more substituents.

4. A compound of claim 3, wherein the substituents include embedded keto, alkenyl, alkynyl, aryl, or heteroaryl groups.

5. A compound of claim 1, wherein R₂ and R₃ are independently selected from the fluorine-containing groups consisting of fluooroalkyl, difluoroalkyl, trifluoroalkyl, polyfluoroalkyl, fluoroaryl, fluoroheteroaryl, fluorocycloalkyl, and fluoroheterocyclic.

6. A method for the synthesis of a compound of claim 1, comprising:
(a) providing an amino acid of formula 30: 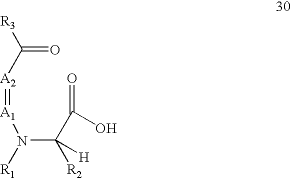
wherein R₁-R₃ and A₁-A₂ are defined as in claim 1; and
(b) reacting the amino acid of formula 30 with an acid activator and a base to form a compound of claim 1.

7. The method of claim 6, wherein the amino acid of compound 30 is converted to an intermediate of formula 31-34, which is transformed to compound 1: 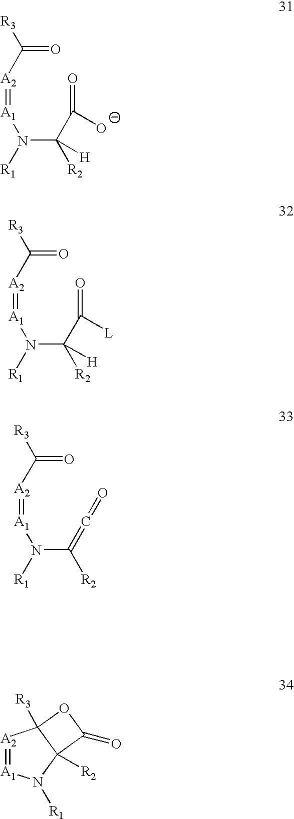
wherein:
R₁-R₃ and A₁-A₂ are defined as in claim 1; and
L is chloro, bromo, iodo, fluoro, OR′, OC(═O)R′, OC(═O)OR′, OC(═O)NR″R′″, OS(═O)R′, OSO₂R′, OPO₂R′, OPO₂OR′, or OP(═O)OR′, wherein R′ is alkyl, fluoroalkyl, aryl, heteroaryl, or 2-N-alkyl-pyridinium, and R″ and R′″ are independently H, alkyl, or aryl.

8. The method of claim 6, wherein the acid activator is selected from the group consisting of an anhydride R^{L}C(═O)—O—C(═O)R^{L}, an acyl fluoride R^{L}C(═O)F, an acyl chloride R^{L}C(═O)Cl, an acyl bromide R^{L}C(═O)Br, a sulfinyl chloride R^{L}S(═O)Cl, a sulfonyl chloride R^{L}SO₂Cl, a sulfinyl anhydride R^{L}S(═O)—O—S(═O)R^{L}, a sulfonyl anhydride R^{L}SO₂—O—SO₂R^{L}, a chloroformate R^{L}OC(═O)Cl, an alkoxyacyl anhydride R^{L}OC(═O)—O—C(═O)OR^{L}, a phosphoryl chloride R^{L}P(═O)Cl, a phosphinyl chloride R^{L}R^{L}P(═O)Cl, a 2-halo-N-alkyl-pyridinium salt, N,N-dimethylphosphoramidic dichloride, thionyl chloride, and oxalyl chloride, wherein R^{L} is methyl, trifluoromethyl, alkyl, fluoroalkyl, difluoroalkyl, trifluoroalkyl, aryl, nitroaryl, or heteroaryl.

9. The method of claim 6, wherein the base is selected from the group consisting of dialkylamine, trialkylamine, and an N-heterocyclic compound containing a basic N-atom.

10. The method of claim 9, wherein the N-heterocyclic compound containing a basic N-atom is selected from the group consisting of pyridine, lutidine, quinoline, isoquinoline, imidazole, diazabicycloundecane (DBU), diazabicyclononane (DBN), and 1,4-diazabicyclo[2.2.2]octane (DABCO).

11. The method of claim 6, wherein the compound synthesized is a compound of formula 4: 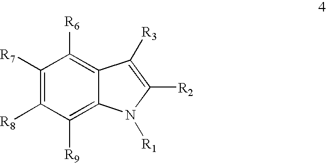
wherein R₁-R₃ and R₆-R₉ are defined as in claim 2.

12. The method of claim 6, wherein the amino acid of formula 30 is prepared in one step by the reaction of an amine compound of formula 35, a boron compound of formula 36 or 37, and glyoxylic acid of formula 38 or its hydrated form: 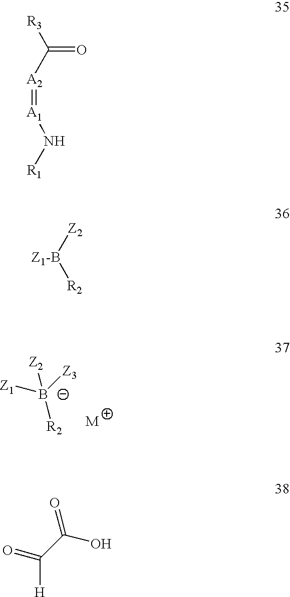
wherein
R₁-R₃ and A₁-A₂ are defined as in claim 1;
Z₁-Z₃ are independently selected from the group consisting of hydroxy, alkoxy, acyloxy, fluoro, chloro, bromo, alkylamino, and arylamino; and
M is potassium or tetralkylamino.

13. The method of claim 12, wherein the amine compound of formula 35 is an aniline.

14. The method of claim 12, wherein the boron compound of formula 36 is an organoboronic acid or boronate.

15. The method of claim 12, wherein the boron compound of formula 37 is an organotrifluoroborate salt.

16. The method of claim 6, wherein at least one of R₂ and R₃ is selected from the fluorine-containing groups consisting of fluooroalkyl, difluoroalkyl, trifluoroalkyl, polyfluoroalkyl, fluoroaryl, fluoroheteroaryl, fluorocycloalkyl, and fluoroheterocyclic.
